(19) United States
(12) Patent Application Publication
(10) Pub. No.: US 2025/0218537 A1
(43) Pub. Date: Jul. 3, 2025

(54) METHODS FOR DIAGNOSING MYOCARDIAL INFARCTION

(71) Applicant: Inflammatix, Inc., Sunnyvale, CA (US)

(72) Inventors: Yudong HE, Kirkland, WA (US); Rushika PANDYA, Austin, TX (US); Timothy E. SWEENEY, Truckee, CA (US); Yehudit HASIN-BRUMSHTEIN, Redwood City, CA (US)

(73) Assignee: Inflammatix, Inc., Sunnyvale, CA (US)

(21) Appl. No.: 18/851,989

(22) PCT Filed: Mar. 6, 2023
(86) PCT No.: PCT/US2023/014600

§ 371 Date: Sep. 27, 2024

Related U.S. Application Data
(60) Provisional application No. 63/325,447, filed on Mar. 30, 2022.

(51) Int. Cl. G16B 25/10; C12Q 1/6883; G16B 40/20; G16H 50/20; G16H 50/70

(52) U.S. Cl. CPC G16B 25/10; C12Q 1/6883; G16B 40/20; G16H 50/20; G16H 50/70; C12Q 2600/158

(57) ABSTRACT

Systems, methods, compositions, apparatuses, and kits for determining whether a subject is undergoing myocardial infarction (MI) using biological samples obtained from the subject, are provided herein. The disclosed methods and compositions involve biomarkers identified from the transcriptomic data of biological samples from subjects with MI. The biomarkers allow the calculation of a composite biomarker value that can be used to determine whether the subject is undergoing MI.

### CROSS-REFERENCES TO RELATED APPLICATIONS

This application is a U.S. national stage filing under 35 U.S.C. § 371 of International Application No. PCT/US2023/014600, filed on Mar. 6, 2023, which claims priority to U.S. Provisional Application No. 63/325,447, filed on Mar. 30, 2022, the disclosure of each of which is hereby incorporated by reference in its entirety for all purposes.

### BACKGROUND

A myocardial infarction (MI), commonly known as a heart attack, occurs when blood flow to the coronary artery of the heart decreases or stops, causing damage to the heart muscle. In a broad term, acute coronary syndrome (ACS), which refers to any constellation of clinical symptoms that may result in acute myocardial ischemia, is among the leading causes of death globally as well as in the US according to reports of WHO and CDC. According to WHO, ischemic heart diseases (IHD) or coronary artery diseases (CAD), despite advances in diagnosis and treatment, continue to be one of the leading causes of mortality across the globe. High mortality rates in addition to financial burden due to acute MI can be significantly reduced with timely and appropriate diagnosis.

A rapid diagnostic test using relevant biomarkers can facilitate an efficient triage of patients with MI or the possibility of MI in case of an inconclusive troponin test and electrocardiogram (ECG, also referred to as EKG) readout. In the initial period of an MI, inconclusive troponin and ECG results can delay critical treatment procedures and increase the burden of patients under observation in the emergency department (ED). A new and accurate diagnostic test that is easy to use in an ED setting and is effective at ruling out the possibility of MI in patients can be immensely beneficial. Rapid and early rule-out of MI can lower the financial and medical costs by avoiding unnecessary hospitalization, expensive investigative procedures, and treatments. Conversely, a quick rule-in of patients undergoing MI would trigger more timely advancement to further tests and interventions, such as surgical procedures, that may be more complex and resource consuming. Reduction of mortality rates may be possible by providing timely intervention.

There is thus a need for new, accurate, rapid, and affordable methods for identifying patients who have MI. The present disclosure satisfies this need and provides other advantages as well.

### SUMMARY

In one aspect, the present disclosure provides a method for a patient suspected of undergoing a myocardial infarction (MI), the method comprising: a) receiving a biological sample obtained from a patient suspected of undergoing the MI; b) measuring one or more expression levels of at least one biomarker selected from Table 2; c) generating a composite biomarker value based on the one or more expression levels of the at least one biomarker in the biological sample; and d) determining, based on the composite biomarker value, whether the patient is undergoing the MI.

A method for a patient suspected of undergoing a myocardial infarction (MI), the method comprising: a) receiving a biological sample obtained from the patient suspected of undergoing the MI; b) measuring one or more expression levels of at least one biomarker selected from Table 2; c) determining whether the patient is undergoing the MI based on the one or more expression levels of the at least one biomarker in the biological sample. In some embodiments, step c) comprises generating a composite biomarker value based on the one or more expression levels of the at least one biomarker in the biological sample, and determining, based on the composite biomarker value, whether the patient is undergoing the MI.

In some embodiments, step b) comprises measuring the expression levels of at least two biomarkers selected from Table 2. In some embodiments, the at least one biomarker is selected from the group consisting of SOCS3, OVCA2, MEF2A, PRR33, SCX, AQP9, IPPK, TNFSF8, CD163, MEF2B, HINT2, ATP1B1, ZCCHC18, CXorf65, and IMP3. In some embodiments, the at least one biomarker is selected from the group consisting of SOCS3, SCX, AQP9, IPPK, CD163, ATP1B1, ZCCHC18, and CXorf65. In some embodiments, the at least one biomarker is selected from the group consisting of OVCA2, MEF2A, PRR33, TNFSF8, MEF2B, HINT2, and IMP3.

In some embodiments of the aforementioned aspects, the composite biomarker value not exceeding a threshold value indicates that the patient is not undergoing the MI, wherein the threshold value is determined using training samples of patients that are determined to not be undergoing the MI by a separate testing procedure. In certain embodiments, the method further comprises the step of determining that the patient is not undergoing the MI by comparing the composite biomarker value to the threshold value. In particular embodiments, the patient is further evaluated by a physician for a condition selected from the group consisting of anxiety disorder, aortic dissection, aortic stenosis, asthma, esophagitis, heart failure, gastroenteritis, left ventricular embolism, musculoskeletal disorder, myocarditis, pericarditis, pneumonia, pulmonary embolism, and unstable angina. In some embodiments, the method can further include the step of discharging the patient from a clinical facility based on determining that the patient is not undergoing the MI.

In some embodiments of the aforementioned aspects, the composite biomarker value exceeding a threshold value indicates that the patient is undergoing the MI and is a candidate for an additional cardiovascular diagnostic testing, a therapeutic intervention, or both, wherein the threshold value is determined using training samples of patients that are determined to be undergoing the MI by a separate testing procedure. In some embodiments, the method further comprises the step of determining that the patient is undergoing the MI by comparing the composite biomarker value to the threshold value and that the patient is a candidate for the additional cardiovascular diagnostic testing, the therapeutic intervention, or both. In some embodiments, the method comprises the step f) of subjecting the patient to the additional cardiovascular diagnostic testing, the therapeutic intervention, or both. In particular embodiments, the additional cardiovascular diagnostic testing comprises one or more of angiography, myocardial perfusion imaging, echocardiography, and magnetic resonance imaging. The angiography can be non-invasive computed tomography (CT) angiography or invasive coronary angiography (ICA). The therapeutic intervention can comprise administration of a pharmaceutical compound, an interventional procedure, or both. The pharmaceutical compound can be an anticoagulant, an antiplatelet, a beta-blocker, a nitrate, a statin, an angiotensin-converting-enzyme (ACE) inhibitor, or an angiotensin receptor blocker (ARBs). The interventional procedure can comprise revascularization (e.g., a percutaneous coronary intervention (PCI) or a coronary artery bypass graft (CABG)).

In some embodiments, the biological sample is obtained in response to the patient experiencing chest pain. In some embodiments, the biological sample is obtained in response to the patient having a cardiac troponin level above a cardiac troponin threshold value. In certain embodiments, the patient has serial cardiac troponin levels above a cardiac troponin level threshold value for at least 3 hours. In certain embodiments, the cardiac troponin threshold value is the 99th percentile upper reference limit value (as described further herein).

In some embodiments of the aforementioned aspects, the MI is ST-elevation myocardial infarction (STEMI). In other embodiments, the MI is non-ST-elevation myocardial infarction (NSTEMI).

In some embodiments, the biological sample is whole blood. In some embodiments, the biological sample is a blood component or a blood fraction such as red blood cells, white blood cells, platelets, peripheral blood mononucleated cells (PBMC), a band cell, a neutrophil, a monocyte, a T cell, or a combination thereof. In some embodiments, the biological sample is serum and/or plasma.

In some embodiments, the expression level of the at least one biomarker is detected using polymerase chain reaction (PCR) (e.g., quantitative PCR (qPCR), droplet digital PCR (ddPCR), reverse transcription PCR (RT-PCR), quantitative RT-PCR (qRT-PCR)), isothermal amplification (e.g., loop-mediated isothermal amplification (LAMP), reverse transcription LAMP (RT-LAMP), quantitative RT-LAMP (qRT-LAMP)), RPA amplification, ligase chain reaction, branched DNA amplification, nucleic acid sequence-based amplification (NASBA), strand displacement assay (SDA), transcription-mediated amplification, rolling circle amplification (RCA), helicase-dependent amplification (HDA), single primer isothermal amplification (SPIA), nicking and extension amplification reaction (NEAR), transcription mediated assay (TMA), CRISPR-Cas detection, and/or direct hybridization without amplification onto a functionalized surface (e.g., using a graphene biosensor).

In some embodiments, the expression level of the at least one biomarker is detected using qRT-PCR. In some embodiments, the expression level of the at least one biomarker is detected using qRT-LAMP.

In another aspect, the disclosure features a test kit for detecting the expression levels of one or more biomarkers in a biological sample of a patient suspected of undergoing a myocardial infarction (MI), wherein the one or more biomarkers comprise at least one biomarker from Table 2.

In certain embodiments of this aspect, the test kit comprises an oligonucleotide for each of the one or more biomarkers, wherein the oligonucleotide hybridizes to the biomarker or a transcript thereof. In some embodiments, the one or more biomarkers are selected from the group consisting of SOCS3, OVCA2, MEF2A, PRR33, SCX, AQP9, IPPK, TNFSF8, CD163, MEF2B, HINT2, ATP1B1, ZCCHC18, CXorf65, and IMP3. The kit can further include, e.g., one or more reagents for performing polymerase chain reaction (PCR) (e.g., reverse transcription PCR (RT-PCR) or loop mediated isothermal amplification (LAMP) (e.g., reverse transcription LAMP (RT-LAMP). The PCR or LAMP can be quantitative.

In some embodiments, the kit is for detecting ST-elevation myocardial infarction (STEMI). In other embodiments, the kit is for detecting non-ST-elevation myocardial infarction (NSTEMI).

The biological sample can be whole blood. In some embodiments, the biological sample is a blood component or a blood fraction such as red blood cells, white blood cells, platelets, peripheral blood mononucleated cells (PBMC), a band cell, a neutrophil, a monocyte, a T cell, or a combination thereof. In some embodiments, the biological sample is serum and/or plasma.

The kit can further include instructions to calculate a composite biomarker value based on the expression levels of the one or more biomarkers in the biological sample of the patient, wherein the composite biomarker value, when compared to a threshold value, indicates the patient undergoing the MI.

In another aspect, the disclosure provides a method for providing an indication for a myocardial infarction (MI) in a subject, the method comprising: (a) measuring expression levels of one or more biomarkers selected from Table 2 or one or more biomarker pairs selected from Table 3 in a biological sample obtained from the subject; (b) evaluating the expression levels of the one or more biomarkers to yield a composite biomarker value, wherein the composite biomarker value exceeding a threshold value indicates that the subject is undergoing the MI; and (c) administering medical care to the subject. In another aspect, the disclosure provides a method for diagnosing a myocardial infarction (MI) in a subject, the method comprising (a) measuring expression levels of one or more biomarkers selected from Table 2 or one or more biomarker pairs selected from Table 3 in a biological sample obtained from the subject; (b) evaluating the expression levels of the one or more biomarkers to yield a composite biomarker value, wherein the composite biomarker value exceeding a threshold value indicates that the subject is undergoing the MI; and (c) administering medical care to the subject.

In some embodiments of the aforementioned aspects, the one or more biomarkers are selected from the group consisting of SOCS3, OVCA2, MEF2A, PRR33, SCX, AQP9, IPPK, TNFSF8, CD163, MEF2B, HINT2, ATP1B1, ZCCHC18, CXorf65, and IMP3.

In some embodiments of the aforementioned aspects, the composite biomarker value has been validated in multiple cohorts. In particular embodiments, an area under the receiver operating characteristic (ROC) curve for the identification of subjects with MI for the composite biomarker value is at least 0.75 in an independent cohort from which the composite biomarker value was derived.

A better understanding of the nature and advantages of embodiments of the present disclosure may be gained with reference to the following detailed description and the accompanying drawings.

### DESCRIPTION OF THE DRAWINGS

FIGS. 1A-1E: Distribution of single gene and 2-gene pair AUCs. AUCs were calculated to evaluate its performance of distinguishing MI samples from other CVD control samples in the 4 studies. FIG. 1A: Background AUC distribution using each of 18,271 mRNAs detected across all 4 studies. FIGS. 1B and 1C: AUCs using each of 337 up- or 143 down-regulated mRNAs identified by absolute effect size 0.6, and FDR value s 0.05. FIG. 1D: AUCs for all 2-gene combinations from 480 mRNA biomarkers. FIG. 1E: AUCs for all 2-gene combinations from randomly sampled 480 mRNA biomarkers from all 18,271 genes.

FIGS. 2A and 2B: Geometric mean value with 480 signature mRNAs distinguishes MI from control samples in all 4 datasets. Geometric mean value is calculated as a scaled difference between the geometric means of expression of up-regulated (n=337) and down-regulated (n=143) mRNAs. FIG. 2A: Boxplot of geometric mean value in MI (label=1) vs control (label=0) shows separation between the two classes in each of the four datasets. FIG. 2B: ROCs and AUCs of such geometric means values in each of the four datasets.

FIGS. 3A and 3B: Geometric mean value with 15 forward search mRNAs distinguishes MI from control samples in all 4 datasets. Geometric mean value is calculated as a scaled difference between the geometric means of expression of up-regulated (n=10) and down-regulated (n=5) mRNAs. FIG. 3A: Boxplot of geometric mean value in MI (label=1) vs control (label=0) shows separation between the two classes in each of the four datasets. FIG. 3B: ROCs and AUCs of such geometric means values based on 15 mRNAs in each of the four datasets.

FIGS. 4A and 4B: The performance of geometric mean value-based classifier. ROCs and AUCs with 480 signature mRNAs (FIG. 4A) and 15 mRNAs obtained from forward search (FIG. 4B) in its discrimination power to distinguish MI from control samples in each and pooled summary of all 4 datasets. In the legend, the point estimate and its 95% confidence level range are given for each and the pooled summary of all 4 datasets.

FIGS. 5A and 5B: The performance of geometric mean value-based classifier for randomly permuted class labels. ROCs and AUCs with the same 480 signature mRNAs (FIG. 5A) and the same 15 forward search mRNAs (FIG. 5B) on the same datasets but for 10 repetitions of randomly permuted class labels. No discriminating power is seen for the permuted labels.

FIG. 6 illustrates a measurement system 600 according to an embodiment of the present disclosure.

FIG. 7 shows a block diagram of an example computer system usable with systems and methods according to embodiments of the present disclosure.

### TERMS

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid” or “polynucleotide” refers to primers, probes, oligonucleotides, template RNA or cDNA, genomic DNA, amplified subsequences of biomarker genes, or any polynucleotide composed of deoxyribonucleic acids (DNA), ribonucleic acids (RNA), or any other type of polynucleotide which is an N-glycoside of a purine or pyrimidine base, or modified purine or pyrimidine bases in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions can be achieved by generating sequences in which the third position of one or more selected (or all) codons is substituted with mixed-base and/or deoxyinosine residues. “Nucleic acid”, “DNA,” “polynucleotides”, and similar terms also include nucleic acid analogs. The polynucleotides are not necessarily physically derived from any existing or natural sequence, but can be generated in any manner, including chemical synthesis, DNA replication, reverse transcription or a combination thereof.

The term “primer” as used herein refers to an oligonucleotide, whether occurring naturally or produced synthetically, that is capable of acting as a point of initiation of synthesis when placed under conditions in which synthesis of a primer extension product which is complementary to a nucleic acid strand is induced, i.e., in the presence of nucleotides (e.g., naturally occurring nucleotides or modified nucleotides), and an agent for polymerization such as DNA polymerase and at a suitable temperature and buffer. Such conditions include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.), and at a suitable temperature. The primer is preferably single-stranded for maximum efficiency in amplification such as a TaqMan real-time quantitative RT-PCR as described herein. Primers may be double-stranded and dissociated into single-strands at a primer melting temperature prior to amplification. The primers herein are selected to be substantially complementary to the different strands of each specific sequence to be amplified, and a given set of primers will act together to amplify a subsequence of the corresponding biomarker gene.

The term “gene” refers to the segment of DNA involved in producing a polypeptide chain. It can include regions preceding and following the coding region (leader and trailer) as well as intervening sequences (introns) between individual coding segments (exons).

The term “myocardial infarction” or “MI” refers to the irreversible necrosis (e.g., death) of heart muscle or cardiac cells secondary to prolonged ischemia that occurs when blood flow decreases or stops to the coronary artery of the heart. This usually results from an imbalance of oxygen supply and demand. One of the most common symptoms of MI is chest pain or discomfort which may travel into other parts of the body, e.g., the shoulder, arm, back, neck, and jaw. In some incidents, the symptoms occur in the center or left side of the chest and last for more than a few minutes. The discomfort may occasionally feel like heartburn. Other symptoms may include, for example, shortness of breath, nausea, feeling faint, a cold sweat, and/or feeling tired. An MI may cause heart failure, an irregular heartbeat, cardiogenic shock, and/or cardiac arrest. Some risk factors of MI include, for example, high blood pressure, smoking, diabetes, lack of exercise, obesity, high blood cholesterol, poor diet, and excessive alcohol intake. Typically, a number of tests are useful to help with diagnosis, including electrocardiograms (ECGs), blood tests (i.e., to test for levels of cardiac enzymes), and coronary angiography. An ECG, which is a recording of the heart's electrical activity, may confirm an ST-elevation MI (STEMI), if ST elevation is present. Commonly used blood tests can test for, for example, levels of troponin and creatine kinase MB. In some cases, the appearance of cardiac enzymes in the circulation can indicate myocardial necrosis.

A “biological sample” refers to a biological specimen obtained from a subject containing, e.g., fluids, cells, or tissues from the subject. For the purposes of the present methods and compositions, a biological sample is taken from a subject suspected of undergoing an MI, and in particular embodiments the sample is whole blood sample or a component of whole blood. Examples of a component of whole blood include, but are not limited to, red blood cells, white blood cells, platelets, peripheral blood mononucleated cells (PBMC), a band cell, a neutrophil, a monocyte, a T cell, or a combination thereof. In other embodiments, the biological sample is serum and/or plasma.

As used herein, a “biomarker gene”, “biomarker mRNA”, or “biomarker” refers to a gene whose expression level in a biological sample of a subject (e.g., a whole blood sample) can be used for diagnosing whether a subject is undergoing an MI. The expression level of each of the genes needs not be correlated with the a patient undergoing MI in all patients; rather, a correlation will exist at the population level, such that the level of expression is sufficiently correlated within the overall population of individuals undergoing MI that it can be combined with the expression levels of other biomarker genes, in any of a number of ways, as described elsewhere herein, and used to calculate a composite biomarker value. The values used for the measured expression level of the individual biomarker genes can be determined in any of a number of ways, including direct readouts from relevant instruments or assay systems or reporter systems, or values determined using methods including, but not limited to, forms of linear or non-linear transformation, rescaling, normalizing, z-scores, ratios against a common reference value, or any other means known to those of skill in the art. In some embodiments, the readout values of the biomarkers are compared to the readout value of a reference or control, e.g., a housekeeping gene whose expression is measured at the same time as the biomarkers. For example, the ratio or log ratio of the biomarkers to the reference gene can be determined. In some embodiments, biomarker genes for the purposes of the present methods include, e.g., SOCS3, OVCA2, MEF2A, PRR33, SCX, AQP9, IPPK, TNFSF8, CD163, MEF2B, HINT2, ATP1B1, ZCCHC18, CXorf65, and IMP3, but others can be used as well, e.g., those presented in Table 2.

The term “composite biomarker value” or “biomarker score” refers to a value allowing a determination of a patient undergoing MI. The composite biomarker value is calculated from the measured expression levels or other readouts of one or a plurality of biomarker genes, e.g., 1, 2, 3, 4, 5, 6, 7, 8, 9, 10, or more individual biomarker genes, in a biological sample (e.g., whole blood sample) from a subject. In some embodiments, the composite biomarker value is determined by applying a mathematical formula, or a series of mathematical formulae with specified interconnections, or a machine learning algorithm with optimized hyperparameters, or another parameter-based method by which the measured expression values of the biomarker genes can be used to generate a single “composite biomarker value”, including, e.g., arithmetic or geometric means with or without weights, linear regression, logistic regression, neural nets, or any other method known in the art. In particular embodiments, the “composite biomarker value” is used to determine whether the patient is undergoing MI by virtue of the composite biomarker value surpassing or not a given threshold value for the outcome in question, as described in more detail elsewhere herein. In some embodiments, the “composite biomarker value” is further converted to a metric value representing the probability of undergoing MI via one or more calibration procedures. The calibration procedure(s) may output a threshold value for diagnosis. In some embodiments, the composite biomarker value is and can be further combined with other factors, such as the presence or severity of specific symptoms, patient factors (e.g. age, sex, vital signs, comorbidities, prior treatment history, or other relevant clinical parameters) to improve the performance or predictive value of the composite biomarker value in determining whether a patient is undergoing MI.

The term “correlating” generally refers to determining a relationship between one random variable with another. In various embodiments, correlating a given composite biomarker value with the presence or absence of a condition or outcome (e.g., undergoing MI) in a subject comprises determining the expression level of at least one biomarker in the subject, using the expression level to calculate a composite biomarker value, and comparing the composite biomarker value with a threshold value, which can be a composite biomarker value from a control subject, such as a healthy subject or a subject who had undergone MI. In specific embodiments, a composite biomarker value calculated based on the expression levels of a set of biomarkers is correlated to the presence or absence of a particular outcome, using receiver operating characteristic (ROC) curves.

### DETAILED DESCRIPTION

The present disclosure provides methods and compositions for determining whether a subject is undergoing myocardial infarction (MI). The present methods and compositions involve biomarkers identified from transcriptomic data of blood samples from individuals with MI and controls, e.g., controls from risk populations (e.g, risk subjects with CAD or stable/unstable angina) or healthy controls. Meta-analysis was used to identify circulating biomarkers from blood that are differentially expressed in the presence of an MI event. 480 mRNAs were identified that can robustly distinguish MI samples from the control samples with a good accuracy suitable for the clinical setting. 15 mRNAs were further down-selected as a parsimonious gene set using a greedy forward search procedure. Diagnostic tests can be established using subsets of the 480 identified mRNA biomarkers. Such diagnostic tests can be utilized in clinics and emergency departments to enable clinicians to make better decisions about whether a subject is undergoing MI and to subsequently employ the appropriate testing and/or therapeutic interventions.

### I. Subjects

The present methods and compositions can be used to determine whether a subject is undergoing MI. In various embodiments, the subject may be an adult of any age, a child, or an adolescent. The subject may be male or female.

In particular embodiments, the subject has one or more of the following symptoms: chest pain or discomfort; feeling weak, light-headed, or faint; developing cold sweat; pain or discomfort in the jaw, neck, and/or back; pain or discomfort in one or both arms and/or shoulders; and/or shortness of breath. In some embodiments, the chest pain or discomfort is in the center or left side of the chest. In some embodiments, the chest pain or discomfort lasts for more than a few minutes (e.g., at least 2, 5, 10, 20, or 30 minutes). In some embodiments, the chest pain or discomfort goes away and comes back. In certain embodiments, the chest pain or discomfort can be uncomfortable pressure, squeezing, fullness, or pain.

In particular embodiments, the subject is present in a medical context, e.g., a clinical setting where diagnosis and/or treatment may take place. A clinical setting does not necessarily indicate that the patient is physically present in a hospital or clinical facility, however. For example, the patient may be at home but has been in communication with a health care provider about his or her condition and its treatment.

The results of the methods described herein can allow a determination of the optimal next step or plan of action for the subject's care. In some embodiments, the determination is that the subject is not undergoing MI. In particular embodiments, the determination is that the subject is not undergoing MI even if the subject presents one or more of the symptoms described above (e.g., chest pain or discomfort). In particular embodiments, the determination is that a subject is undergoing MI. In certain embodiments, the determination is that the subject is undergoing MI when the subject presents one or more of the symptoms described above (e.g., chest pain or discomfort).

### II. Biological Samples

To assess the biomarker status of the subject, a biological sample is obtained. In some embodiments, the sample is a blood sample, e.g., plasma, serum, or whole blood sample. In particular embodiments, the sample is a whole blood sample obtained from the subject. In some embodiments, the biological sample is a blood component such as red blood cells, white blood cells, platelets, peripheral blood mononucleated cells (PBMC), a band cell, a neutrophil, a monocyte, a T cell, or a combination thereof. In some embodiments, the biological sample is serum and/or plasma.

Other potential samples that can be used include, urine, ascites, seminal fluid, vaginal secretions, cerebrospinal fluid (CSF), synovial fluid, pleural fluid (pleural lavage), pericardial fluid, peritoneal fluid, amniotic fluid, saliva, nasal fluid, otic fluid, gastric fluid, breast milk, amniotic fluid, bile, gastric juice, lymph, mucus, pericardial fluid, peritoneal fluid, pleural fluid, pus, saliva, sebum, serous fluid, sputum, sweat, tears, and others. The biological sample, e.g., a whole blood sample, can be obtained from the subject using conventional techniques known in the art. In some embodiments, the sample is obtained for the purposes of assessing the subject's biomarker status using the herein-described methods.

In some embodiments, the biological sample, e.g., a whole blood sample, can be obtained at different times from the subject. For example, the biological sample, e.g., a whole blood sample or a component thereof, can be obtained in response to the subject having a cardiac troponin level above a cardiac troponin threshold value (e.g., a cardiac troponin threshold value is the 99th percentile upper reference limit value). In particular embodiments, the subject has serial cardiac troponin levels above a cardiac troponin level threshold value (e.g., a cardiac troponin threshold value is the 99th percentile upper reference limit value) over the course of at least 3 hours (e.g., at least 4, 5, 6, 7, 8, 9, or 10 hours). In some embodiments, cardiac troponin threshold values can be determined using commercially available troponin tests. Some examples of commercially available tests and their 99th percentile troponin values include, e.g., ARCHITECT STAT High Sensitive Troponin-I by Abbott (99th percentile troponin value at 15.6 ng/L for female and 34.2 ng/L for male); Access hsTnI by Beckman Coulter (99th percentile troponin value at 11.6 ng/L for female and 19.8 ng/L for male); VIDAS High Sensitive Troponin I by bioMérieux (99th percentile troponin value at 11 ng/L for female and 25 ng/L for male); Pylon hsTnI assay by ET Healthcare (99th percentile troponin value at 21 ng/L for female and 27 ng/L for male); Pylon hsTnT by ET Healthcare (99th percentile troponin value at 13 ng/L for female and 14 ng/L for male); Lumipulse G G1200 and G60011 hsTnI by Fujirebio (99th percentile troponin value at 22.4 ng/L for female and 32.9 ng/L for male); cobas e601, e602, E170/TnT Gen 5 STAT by Roche (99th percentile troponin value at 14 ng/L for female and 22 ng/L for male); ATELLICA High-Sensitivity TnI (TnIH) by Siemens (99th percentile troponin value at 38.6 ng/L for female and 53.5 ng/L for male); and Singulex Clarity cTnI (99th percentile troponin value at 8.76 ng/L for female and 9.23 ng/L for male). Other methods of determining troponin levels and troponin threshold values are described in, e.g., Apple, Clin Chem 2010; 56:886-91; Apple and Collinson, Clin Chem 2012; 58:54-61; Wu and Christenson, Clin Biochem 2013; 46:969-78.; and Wu et al., Clin Chem 2009; 55:52-8.

In another example, the biological sample, e.g., a whole blood sample or component thereof, can be obtained from the subject before, during, and/or after the subject is actively experiencing one or more of the symptoms described above, e.g., chest pain or discomfort. In certain embodiments, the biological sample, e.g., a whole blood sample, can be obtained from the subject minutes or hours (e.g., 5, 10, 20, 30, 40, or 50 minutes; e.g., 1, 2, 3, 4, or 5 hours) before the subject is actively experiencing one or more of the symptoms described above, e.g., chest pain or discomfort. In certain embodiments, the biological sample, e.g., a whole blood sample, can be obtained from the subject minutes or hours (e.g., 5, 10, 20, 30, 40, or 50 minutes; e.g., 1, 2, 3, 4, or 5 hours) after the subject has experienced one or more of the symptoms described above, e.g., chest pain or discomfort. In certain embodiments, the biological sample, e.g., a whole blood sample or component thereof, can be obtained from the subject while the subject is actively experiencing one or more of the symptoms described above, e.g., chest pain or discomfort. The biological sample, e.g., a whole blood sample, can be obtained by the same caregiver or clinical facility as that carrying out the herein-described methods, or can be obtained from a different source (e.g., different caregiver or clinical facility).

### III. Methods and Selection of Biomarkers

As described herein, the methods for a subject suspected of undergoing a myocardial infarction (MI) can include: a) receiving a biological sample obtained from a subject suspected of undergoing the MI; b) measuring one or more expression levels of at least one biomarker selected from Table 2; c) generating a composite biomarker value based on the one or more expression levels of the at least one biomarker in the sample; and d) determining, based on the composite biomarker value, whether the subject is undergoing the MI. In some embodiments of the methods, the composite biomarker value exceeding a threshold value indicates that the subject is undergoing the MI and is a candidate for an additional cardiovascular diagnostic testing, a therapeutic intervention, or both. In some embodiments, the threshold value is determined using training samples of subjects that are determined to be undergoing the MI by a separate testing procedure. The methods can further comprise the step e) of determining that the subject is undergoing the MI by comparing the composite biomarker value to the threshold value and that the subject is a candidate for the additional cardiovascular diagnostic testing, the therapeutic intervention, or both. The methods can further comprise the step f) of subjecting the subject to the additional cardiovascular diagnostic testing, the therapeutic intervention, or both. The additional cardiovascular diagnostic testing can comprise one or more of angiography, myocardial perfusion imaging, echocardiography, and magnetic resonance imaging. As used herein, the terms “composite biomarker value” and “biomarker score” are interchangeable.

In some embodiments, the MI is ST-elevation myocardial infarction (STEMI). In some embodiments, the MI is non-ST-elevation myocardial infarction (NSTEMI).

The determination that the subject is undergoing MI can be determined by calculating a composite biomarker value based on the expression levels of biomarkers in a biological sample, e.g., a whole blood sample or component thereof, obtained from the subject. In some embodiments, a panel of several biomarkers is used to calculate the composite biomarker value. For example, in some embodiments, biomarkers used in the methods include, but are not limited to, any one or more of the 480 biomarkers listed in Table 2. Any number of total biomarkers can be selected from the 480 biomarkers (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 90, 95, 100, 200, 300, or 400 biomarkers) listed in Table 2 and be used to generate the composite biomarker value. In some embodiments, the biomarkers include any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more biomarkers listed in Table 2.

In some embodiments, the biomarkers include any one or more pairs of biomarkers that can be generated from the 480 biomarkers listed in Table 2. For example, in some embodiments, the biomarkers include any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more pairs of biomarkers that can be generated from the 480 biomarkers listed in Table 2.

In some embodiments, the biomarkers include any one or more pairs of biomarkers listed in Table 3. For example, in some embodiments, the biomarkers include any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 pairs of biomarkers listed in Table 3. In particular embodiments, the composite biomarker value is calculated based on the expression levels of any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 pairs of biomarkers listed in Table 3.

In some embodiments, a biomarker used in methods described herein is selected from the group consisting of SOCS3, OVCA2, MEF2A, PRR33, SCX, AQP9, IPPK, TNFSF8, CD163, MEF2B, HINT2, ATP1B1, ZCCHC18, CXorf65, and IMP3. In some embodiments, at least 2, 3, 4, 5, 6, 7, 8, 9, 10, 11, 12, 13, 14, or 15 biomakers selected from the group consisting of SOCS3, OVCA2, MEF2A, PRR33, SCX, AQP9, IPPK, TNFSF8, CD163, MEF2B, HINT2, ATP1B1, ZCCHC18, CXorf65, and IMP3 are used in methods described herein. In particular embodiments, one or more pairs of biomarkers selected from the group consisting of SOCS3, OVCA2, MEF2A, PRR33, SCX, AQP9, IPPK, TNFSF8, CD163, MEF2B, HINT2, ATP1B1, ZCCHC18, CXorf65, and IMP3 are used in methods described herein.

In some embodiments, a biomarker used in methods described herein is selected from the group consisting of SOCS3, SCX, AQP9, IPPK, CD163, ATP1B1, ZCCHC18, and CXorf65. In some embodiments, at least 2, 3, 4, 5, 6, 7, or 8 biomakers selected from the group consisting of SOCS3, SCX, AQP9, IPPK, CD163, ATP1B1, ZCCHC18, and CXorf65 are used in methods described herein. In particular embodiments, one or more pairs of biomarkers selected from the group consisting of SOCS3, SCX, AQP9, IPPK, CD163, ATP1B1, ZCCHC18, and CXorf65 are used in methods described herein.

In some embodiments, a biomarker used in methods described herein is selected from the group consisting of OVCA2, MEF2A, PRR33, TNFSF8, MEF2B, HINT2, and IMP3. In some embodiments, at least 2, 3, 4, 5, 6, or 7 biomakers selected from the group consisting of OVCA2, MEF2A, PRR33, TNFSF8, MEF2B, HINT2, and IMP3 are used in methods described herein. In particular embodiments, one or more pairs of biomarkers selected from the group consisting of OVCA2, MEF2A, PRR33, TNFSF8, MEF2B, HINT2, and IMP3 are used in methods described herein.

The biomarkers used in the present methods correspond to genes whose expression levels in blood are differentially expressed in the presence of an MI event. The expression level of the individual biomarkers can be elevated or depressed in subjects undergoing MI. For example, in particular embodiments, the expression level of at least one biomarker (e.g., 2, 3, 4, 5, 6, 7, or 8) of SOCS3, SCX, AQP9, IPPK, CD163, ATP1B1, ZCCHC18, and CXorf65 of a subject undergoing MI is reduced relative to a reference expression level of the same biomarker of a control subject (i.e., a subject who is not undergoing MI). In particular embodiments, the composite biomarker value generated from the expression level of at least one biomarker (e.g., 2, 3, 4, 5, 6, 7, or 8) of SOCS3, SCX, AQP9, IPPK, CD163, ATP1B1, ZCCHC18, and CXorf65 of a subject undergoing MI exceeds a threshold value.

In another example, in particular embodiments, the expression level of at least one biomarker (e.g., 2, 3, 4, 5, 6, or 7) of OVCA2, MEF2A, PRR33, TNFSF8, MEF2B, HINT2, and IMP3 of a subject undergoing MI is increased relative to a reference expression level of the same biomarker of a control subject (i.e., a subject who is not undergoing MI). In particular embodiments, the composite biomarker value generated from the expression level of at least one biomarker (e.g., 2, 3, 4, 5, 6, or 7) of OVCA2, MEF2A, PRR33, TNFSF8, MEF2B, HINT2, and IMP3 of a subject undergoing MI exceeds a threshold value. The expression level of a biomarker can be positively or inversely correlated with the determination that the subject is undergoing MI, allowing the determination of an overall composite biomarker value that can be used to inform a diagnostic or treatment decision.

In some embodiments of the methods described herein, the composite biomarker value not exceeding a threshold value indicates that the subject is not undergoing the MI. The methods can further comprise the step e) of determining that the subject is not undergoing the MI by comparing the composite biomarker value to the threshold value. Once it is determined that the subject is not undergoing MI, the subject can be further evaluated by a physician for a condition selected from the group consisting of anxiety disorder, aortic dissection, aortic stenosis, asthma, esophagitis, heart failure, gastroenteritis, left ventricular embolism, musculoskeletal disorder, myocarditis, pericarditis, pneumonia, pulmonary embolism, and unstable angina. Further, the methods can include the step of discharging the subject from a clinical facility based on determining the subject is not undergoing the MI.

Additional biomarkers that can be used in the present methods can be assessed and identified using any standard analysis method or metric, e.g., by analyzing data from samples taken from subjects who are undergoing MI or who have undergone MI. Suitable metrics and methods include Pearson correlation, Kendall rank correlation, Spearman rank correlation, t-test, other non-parametric measures, linear regression, non-linear regression, random forest and other tree-based methods, artificial neural networks, etc. In one embodiment, the feature selection uses univariate ranking with the absolute value of the Pearson correlation between the gene expression and outcome as the ranking metric. In some embodiments, features (genes) are selected using metrics that measure the effect size between different groups of subjects, i.e., subjects with MI and controls, e.g., controls from risk populations (e.g, risk subjects with CAD or stable/unstable angina). In some embodiments, features (genes) are further selected via greedy forward search optimized on training accuracy for a parsimonious set of genes. In some embodiments, features (genes) are selected via greedy forward search optimized on Area Under Operator Receiver Characteristic.

In some embodiments, data from multiple sources is inputted to a multi-cohort analysis using appropriate software, e.g., the Metalntegrator package. In some embodiments, effect size is calculated for each mRNA within a study between different groups of subjects, i.e., subjects with MI and controls, e.g., controls from risk populations (e.g, risk subjects with CAD or stable/unstable angina), e.g., as Hedges' g. In some embodiments, the pooled or summary effect size across all of the datasets is then computed, e.g., using DerSimonian and Laird's random effects model. In some embodiments, the effect size is then summarized and p values across all mRNAs corrected for multiple testing, e.g., based on Benjamini-Hochberg false discovery rate (FDR). In some embodiments, the p-values across the studies are then combined, e.g., using Fisher's sum of logs method, and the log-sum of p values that each mRNA is up- or downregulated is computed, along with corresponding p values. In some embodiments, meta-analysis is performed, e.g., by performing leave one-study out (LOO) analysis by removing one dataset at a time. In some embodiments, a greedy forward search can be used to identify a parsimonious set of genes with the greatest discriminatory power to distinguish samples from different groups of subjects, i.e., subjects with MI and controls, e.g., controls from risk populations (e.g, risk subjects with CAD or stable/unstable angina).

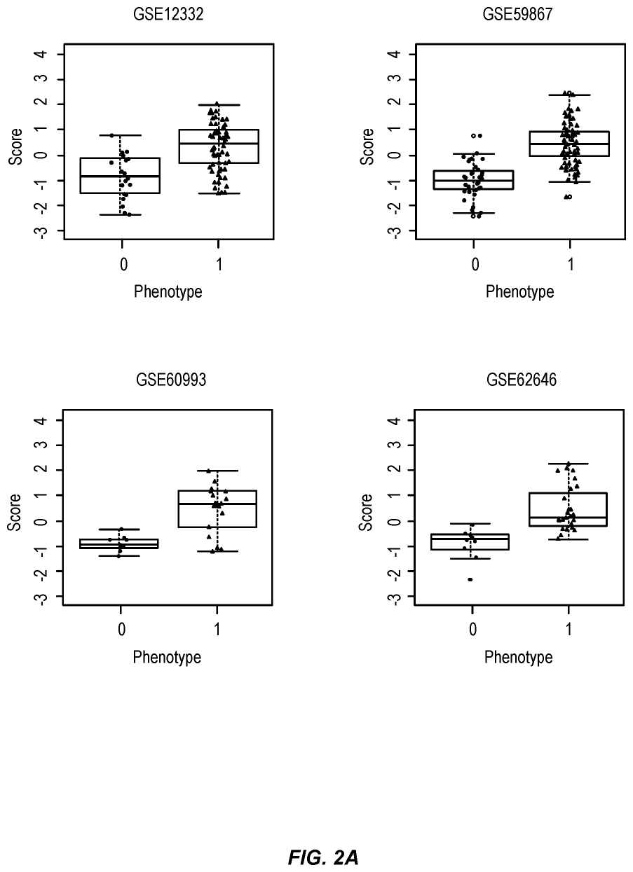

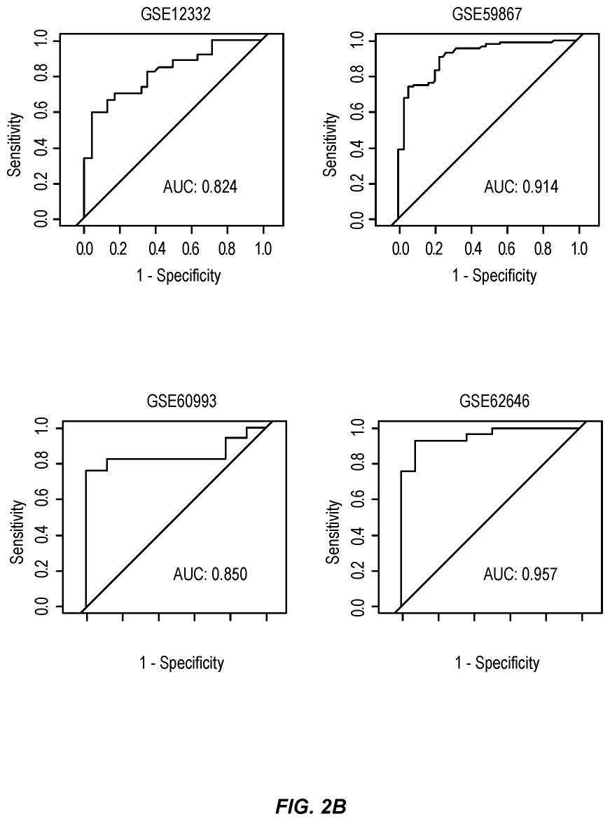

In particular embodiments, a machine learning workflow is applied to the training data, e.g., using a separate validation set or using cross-validation. For example, hyperparameter tuning can be used over a search space of parameters, e.g., parameters known to be effective for model optimization for infectious disease diagnosis. Examples of classifiers that can be used include linear classifiers such as Support Vector Machine (SVM) with linear kernel, logistic regression, and multi-layer perceptron with linear activation function, and non-linear classifiers such as SVM with non-linear kernel. Feature selection can be performed using the gene expression data for the candidate biomarkers as independent variables and using the known outcome as the dependent variable. The different models can be evaluated, e.g., using plots based on sensitivity and false-positive rates for each model, and the decision threshold evaluated during the hyperparameter search, and using ROC-like plots based on pooled cross-validated probabilities for the best models. (See, e.g., Ramkumar et al., Development of a Novel Proteomic Risk-Classifier for Prognostication of Patients with Early-Stage Hormone Receptor-Positive Breast Cancer. Biomarker Insights, Vol. 13, 1-9, 2018, FIG. 2A). Any of a number of different variants of cross-validation (CV) can be used, such as 5-fold random CV, 5-fold grouped CV, where each fold comprises multiple studies, and each study is assigned to exactly one CV fold, and leave-one-study-out (LOSO), where each study forms a CV fold. In some embodiments, the number of genes included in the final model can be limited, e.g., to 5, 6, 7, or 8, to facilitate translation to a rapid molecular assay.

### IV. Detecting Biomarker Expression

As described in more detail below, data sets corresponding to the biomarker expression levels as described herein are used to create a composite biomarker value. The expression levels of the biomarkers can be assessed in any number of ways. In particular embodiments, the expression levels of the biomarkers are determined by measuring polynucleotide levels of the biomarkers. For example, once the biological sample (e.g., a whole blood sample or component thereof) has been collected and preserved, RNA can be extracted using any method, so long that it permits the preservation of the RNA for subsequent quantification of the expression levels of the biomarker genes and of any control genes to be used, e.g., housekeeping genes used as reference values for the biomarkers. RNA can be extracted, e.g., from preserved cells manually, or using a robotic apparatus, such as Qiacube (QIAGEN) with a commercial RNA extraction kit. In some embodiments, RNA extraction is not performed, e.g., for isothermal amplification methods. In such methods, expression levels can be determined directly through lysis of cells, and then, e.g., reverse transcription and amplification of mRNA.

In some embodiments, the reference nucleic acid is a housekeeping gene or a product thereof, such as a corresponding mRNA transcript. In some embodiments, the reference nucleic acid includes an mRNA transcript that is a pre-mRNA molecule, a 5′ capped mRNA molecule, a 3′ adenylated mRNA molecule, or a mature mRNA molecule. In particular embodiments, the reference nucleic acid is a mature mRNA molecule obtained from a mammalian host that is also the source of the test sample. In some embodiments, the housekeeping gene or product thereof is expressed at a relatively constant rate by a cell of the host, such that the expression rate of the housekeeping gene can be used as a reference point against the expression of other host genes or gene products thereof.

Exemplary human housekeeping genes suitable for use with the present methods include, but are not limited to, YWHAB, Chromosome 1 open reading frame 43 (C1orf43), Charged multivesicular body protein 2A (CHMP2A), ER membrane protein complex subunit 7 (EMC7), Glucose-6-phosphate isomerase (GPI), Proteasome subunit, beta type, 2 (PSMB2), Proteasome subunit, beta type, 4 (PSMB4), Member RAS oncogene family (RAB7A), Receptor accessory protein 5 (REEP5), small nuclear ribonucleoprotein D3 (SNRPD3), Valosin containing protein (VCP) and vacuolar protein sorting 29 homolog (VPS29). In some embodiments, any housekeeping gene provided at www/tau/ac/il˜elieis/HKG/may be used (see, Eisenberg and Levanon., Trends Genet. (2013), 10:569-74). Other suitable housekeeping genes include, e.g., GAPDH, ubiquitin, 18S (18S rRNA, e.g., HGNC (Human Genome Nomenclature Committee) nos. 44278-44281, 37657), ACTB (Actin beta, e.g., HGNC no. 132)), KPNA6 (Karyopherin subunit alpha 6, e.g., HGNC no. 6399), or RREB1 (ras-responsive element binding protein 1, e.g., HGNC no. 10449).

The levels of transcripts of the biomarker genes, or their levels relative to one another, and/or their levels relative to a reference gene such as a housekeeping gene, can be determined from the amount of mRNA, or polynucleotides derived therefrom, present in a biological sample. Polynucleotides can be detected and quantified by a variety of methods including, but not limited to, NanoString (e.g., nCounter analysis), microarray analysis, polymerase chain reaction (PCR) (e.g., quantitative PCR (qPCR), droplet digital PCR (ddPCR), reverse transcriptase polymerase chain reaction (RT-PCR), quantitative RT-PCR (qRT-PCR)), isothermal amplification (e.g., loop-mediated isothermal amplification (LAMP), reverse transcription LAMP (RT-LAMP), quantitative RT-LAMP (qRT-LAMP)), RPA amplification, ligase chain reaction, branched DNA amplification, nucleic acid sequence-based amplification (NASBA), strand displacement assay (SDA), transcription-mediated amplification, rolling circle amplification (RCA), helicase-dependent amplification (HDA), single primer isothermal amplification (SPIA), nicking and extension amplification reaction (NEAR), transcription mediated assay (TMA), CRISPR-Cas detection, direct hybridization without amplification onto a functionalized surface (e.g., graphene biosensor), serial analysis of gene expression (SAGE), internal DNA detection switch, northern blotting, RNA fingerprinting, sequencing methods, Qbeta replicase, strand displacement amplification, transcription based amplification systems, nuclease protection (Si nuclease or RNAse protection assays), as well as methods disclosed in International Publication Nos. WO 88/10315 and WO 89/06700, and International Applications Nos. PCT/US87/00880 and PCT/US89/01025; herein incorporated by reference in their entireties, and methods using MacMan probes, flip probes, and TaqMan probes (see, e.g., Murray et al. (2014) J. Mol Diag. 16:6, pp 627-638). See, e.g., Draghici, Data Analysis Tools for DNA Microarrays, Chapman and Hall/CRC, 2003; Simon et al., Design and Analysis of DNA Microarray Investigations, Springer, 2004; Real-Time PCR: Current Technology and Applications, Logan, Edwards, and Saunders eds., Caister Academic Press, 2009; Bustin, A-Z of Quantitative PCR (IUL Biotechnology, No. 5), International University Line, 2004; Velculescu et al. (1995) Science 270: 484-487; Matsumura et al. (2005) Cell. Microbiol. 7: 11-18; Serial Analysis of Gene Expression (SAGE): Methods and Protocols (Methods in Molecular Biology), Humana Press, 2008; each of which is herein incorporated by reference in its entirety.

In some embodiments, the biomarker gene expression is detected using a gene expression panel such as a NanoString nCounter, which allows the quantification of biomarker gene expression without the need for amplification or cDNA conversion. In such methods, RNA obtained from the blood or other biological sample from the subject is hybridized in solution to probes, e.g., a labeled reporter probe and a capture probe for each biomarker and control sequence. The target RNA-probe complexes are then purified and immobilized on a solid support, and then quantified, with each marker-specific probe having a specific fluorescent signature that allows the quantification of the specific marker. Such methods and the generation of probes, e.g., capture probes and reporter probes, for such applications are known in the art and are described, e.g., on the website nanostring.com.

For amplification-based methods such as qRT-PCR or qRT-LAMP, the primers can be obtained in any of a number of ways. For example, primers can be synthesized in the laboratory using an oligo synthesizer, e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc.

Both naturally occurring nucleotides, modified nucleotides, as well as labeled nucleotides, can be used in methods such as qRT-PCR and qRT-LAMP. A modified nucleotide can have a modified nucleobase, a modified sugar portion, and/or a modified internucleotisde linkage. A modified nucleobase (or base) refers to a nucleobase having at least one change that is structurally distinguishable from a naturally-occurring nucleobase (e.g., adenine, guanine, cytosine, thymine, or uracil). In some embodiments, a modified nucleobase is functionally interchangeable with its naturally-occurring counterpart. Both naturally-occurring and modified nucleobases are capable of hydrogen bonding. Modified nucleobases may help to improve the stability of a polynucleotide, such as increasing its half-life and preventing intracellular degradation and proteolytic cleavage. Examples of modified nucleobases include, but are not limited to, 5-methylcytosine, 5-hydroxymethyl cytosine, xanthine, hypoxanthine, 2-aminoadenine, 6-methyladenine, 6-methylguanine, 2-propyladenine, 2-propylguanine, 2-thiouracil, 2-thiothymine, 2-thiocytosine, 5-halouracil, 5-halocytosine, 5-propynyluracil, 5-propynylcytosine, 6-azouracil, 6-azocytosine, 6-azothymine, 5-uracil (pseudouracil), 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-halouracil, 5-bromouracil, 5-trifluoromethyluracil, 5-halocytosine, 5-bromocytosine, 5-trifluoromethylcytosine, 7-methylguanine, 7-methyladenine, 2-fluoroadenine, 2-aminoadenine, 8-azaguanine, 8-azaadenine, 7-deazaguanine, 7-deazaadenine, 3-deazaguanine, and 3-deazaadenine.

A modified sugar refers to a sugar having at least one change that is structurally distinguishable from a naturally-occurring sugar (e.g., deoxyribose in DNA or ribose in RNA). Modifications on modified sugars may help to improve the stability of a polynucleotide. In some embodiments, the sugar is a pentofuranosyl sugar. The pentofuranosyl sugar ring of a nucleoside may be modified in various ways including, but not limited to, addition of a substituent group, particularly, at the 2′ position of the ring; bridging two non-geminal ring atoms to form a bicyclic sugar (i.e., a locked sugar); and substitution of an atom or group such as —S—, —N(R)— or —C(R1)(R2) for the ring oxygen. Examples of modified sugars include, but are not limited to, substituted sugars, especially 2′-substituted sugars having a 2′-F, 2′-OCH2 (2′-OMe), or a 2′-O(CH2)2-OCH3 (2′-O-methoxyethyl or 2′-MOE) substituent group; and bicyclic sugars. A bicyclic sugar refers to a modified pentofuranosyl sugar containing two fused rings. For example, a bicyclic sugar may have the 2′ ring carbon of the pentofuranose linked to the 4′ ring carbon by way of one or more carbons (i.e., a methylene) and/or heteroatoms (i.e., sulfur, oxygen, or nitrogen). The second ring in the sugar limits the flexibility of the sugar ring and thus, constrains the oligonucleotide in a conformation that is favorable for base pairing interactions with its target nucleic acids. An example of a bicyclic sugar is a locked sugar, which is a pentofuranosyl sugar having the 2′-oxygen linked to the 4′ ring carbon by way of a carbon (i.e., a methylene) or a heteroatom (i.e., sulfur, oxygen, or nitrogen). In some embodiments, a locked sugar has the 2′-oxygen linked to the 4′ ring carbon by way of a carbon (i.e., a methylene). In other words, a locked sugar has a 4′-(CH2)-O-2′ bridge, such as α-L-methyleneoxy (4′-CH2-O-2′) and β-D-methyleneoxy (4′-CH2-O-2′). A nucleoside having a lock sugar is referred to as a locked nucleoside.

Other examples of bicyclic sugars include, but are not limited to, (6′S)-6′ methyl bicyclic sugar, aminooxy (4′-CH2-O—N(R)-2′) bicyclic sugar, oxyamino (4′-CH2-N(R)—O-2′) bicyclic sugar, wherein R is, independently, H, a protecting group or C1-C12 alkyl. The substituent at the 2′ position can also be selected from allyl, amino, azido, thio, O-allyl, O—C1-C10 alkyl, OCF3, O(CH2)2SCH3, O(CH2)2-O—N(Rm)(Rn), and O—CH2-C(═O)—N(Rm)(Rn), wherein each Rm and Rn is, independently, H or substituted or unsubstituted C1-C10 alkyl. In some embodiments, a modified sugar is an unlocked sugar. An unlocked sugar refers to an acyclic sugar that has a 2′, 3′-seco acyclic structure, where the bond between the 2′ carbon and the 3′ carbon in a pentofuranosyl ring is absent.

In other embodiments, a modified nucleotide can contain a naturally occurring or a modified internucleoside linkage or phosphate backbone. An internucleoside linkage refers to the backbone linkage that connects the nucleosides. An internucleoside linkage may be a naturally-occurring internucleoside linkage (i.e., a phosphate linkage, also referred to as a 3′ to 5′ phosphodiester linkage, which is found in DNA and RNA) or a modified internucleoside linkage. A modified internucleoside linkage refers to an internucleoside linkage having at least one change that is structurally distinguishable from a naturally-occurring internucleoside linkage. Modified internucleoside linkages may help to improve the stability of a polynucleotide. Examples of modified internucleoside linkages include, but are not limited to, a phosphorothioate linkage, a phosphorodithioate linkage, a phosphoramidate linkage, a phosphorodiamidate linkage, a thiophosphoramidate linkage, a thiophosphorodiamidate linkage, a phosphoramidate morpholino linkage, and a thiophosphoramidate morpholino linkage, and a thiophosphorodiamidate morpholino linkage, which are known in the art and described in, e.g., Bennett and Swayze, Annu Rev Pharmacol Toxicol. 50:259-293, 2010. A phosphorothioate linkage is a 3′ to 5′ phosphodiester linkage that has a sulfur atom for a non-bridging oxygen in the phosphate backbone of an oligonucleotide. A phosphorodithioate linkage is a 3′ to 5′ phosphodiester linkage that has two sulfur atoms for non-bridging oxygens in the phosphate backbone of an oligonucleotide. A thiophosphoramidate linkage refers to a 3′ to 5′ phospho-linkage that has a sulfur atom for a non-bridging oxygen and a NH group as the 3′-bridging oxygen in the phosphate backbone of an oligonucleotide.

Computer programs can be used in the design of primers with the required specificity and optimal amplification properties, such as Oligo version 5.0 (National Biosciences). PCR methods can also be implemented.

In some embodiments, microarrays are used to measure the levels of biomarkers. An advantage of microarray analysis is that the expression of each of the biomarkers can be measured simultaneously, and microarrays can be specifically designed to provide a diagnostic expression profile for a particular disease or condition. Microarrays are prepared by selecting probes which comprise a polynucleotide sequence, and then immobilizing such probes to a solid support or surface. For example, the microarray may comprise a support or surface with an ordered array of binding (e.g., hybridization) sites or “probes” each representing one of the biomarkers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). Each probe is preferably covalently attached to the solid support at a single site. Conditions for preparing microarrays, for hybridization conditions, and for detection of bound probes can be implemented.

In some embodiments, RNA sequencing (RNA-seq) can be used to measure the expression levels of biomarkers. RNA-seq is a technique based on enumeration of RNA transcripts using next-generation sequencing methodologies. In general, a population of RNA (total or fractionated, such as poly(A)+) is converted to a library of cDNA fragments with adaptors attached to one or both ends. Each molecule, with or without amplification, is then sequenced in a high-throughput manner to obtain short sequences from one end (single-end sequencing) or both ends (pair-end sequencing). The reads are typically 30-400 bp, depending on the DNA-sequencing technology used. Any high-throughput sequencing technology can be used for RNA-Seq, such as the Illumina IG, Applied Biosystems SOLiD, and Roche 454 Life Science systems. The Helicos Biosciences tSMS system has the added advantage of avoiding amplification of target cDNA. Following sequencing, the resulting reads are either aligned to a reference genome or reference transcripts, or assembled de novo without the genomic sequence to produce a genome-scale transcription map that consists of both the transcriptional structure and/or level of expression for each gene.

As noted above, the “probe” to which a particular polynucleotide molecule specifically hybridizes contains a complementary polynucleotide sequence. The probes of the microarray typically consist of nucleotide sequences of, e.g., no more than 1,000 nucleotides, or of 10 to 1,000 nucleotides or 10-200, 10-30, 10-40, 20-50, 40-80, 50-150, or 80-120 nucleotides in length. The probes may comprise DNA sequences, RNA sequences, or copolymer sequences of DNA and RNA. The polynucleotide sequences of the probes may also comprise DNA and/or RNA analogs, derivatives, or combinations thereof. For example, the probes can be modified at the base moiety, at the sugar moiety, or at the phosphate backbone (e.g., phosphorothioates). The polynucleotide sequences of the probes may be synthesized nucleotide sequences, such as synthetic oligonucleotide sequences. The probe sequences can be synthesized either enzymatically in vivo, enzymatically in vitro (e.g., by PCR), or non-enzymatically in vitro.

Probes are preferably selected using an algorithm that takes into account binding energies, base composition, sequence complexity, cross-hybridization potential based on probe similarities with other genes in the genome, and secondary structure. See Friend et al., International Patent Publication WO 01/05935, published Jan. 25, 2001; Hughes et al., Nat. Biotech. 19:342-7 (2001). An array will include both positive control probes, e.g., probes known to be complementary and hybridizable to sequences in the target polynucleotide molecules, and negative control probes, e.g., probes known to not be complementary and hybridizable to sequences in the target polynucleotide molecules. In addition, the present methods will include probes to both the biomarkers themselves, as well as to internal control sequences such as housekeeping genes, as described in more detail elsewhere herein.

In some embodiments, quantitative reverse transcriptase PCR (qRT-PCR) is used to determine the expression profiles of biomarkers (see, e.g., U.S. Patent Application Publication No. 2005/0048542A1; herein incorporated by reference in its entirety). The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

In some embodiments, the PCR employs the Taq DNA polymerase, which has a 5-3′ nuclease activity but lacks a 3-5′ proofreading endonuclease activity. TAQMAN PCR typically utilizes the 5′-nuclease activity of Taq or Tth polymerase to hydrolyze a hybridization probe bound to its target amplicon, but any enzyme with equivalent 5′ nuclease activity can be used. In such methods, two oligonucleotide primers are used to generate an amplicon typical of a PCR reaction, and a third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN RT-PCR can be performed using commercially available equipment, such as, for example, ABI PRISM 7700 sequence detection system. (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 sequence detection system. The system consists of a thermocycler, laser, charge-coupled device (CCD), camera and computer. The system includes software for running the instrument and for analyzing the data. 5′-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs that can be used to normalize patterns of gene expression include mRNAs for the housekeeping genes glyceraldehyde-3-phosphate-dehydrogenase (GAPDH) and beta-actin.

In particular embodiments, the biomarker gene expression is determined using isothermal amplification. Isothermal amplification is a process in which a target nucleic acid is amplified using a constant, single, amplification temperature (e.g., from about 30° C. to about 95° C.). Unlike standard PCR, an isothermal amplification reaction does not include multiple cycles of denaturation, hybridization, and extension, of an annealed oligonucleotide to form a population of amplified target nucleic molecules (i.e., amplicons). There are various types of isothermal application known in the art, including but not limited to, loop-mediated isothermal amplification (LAMP), nucleic acid sequence based amplification NASBA, recombinase polymerase amplification (RPA), rolling circle amplification (RCA), nicking enzyme amplification reaction (NEAR), and helicase dependent amplification (HDA).

In particular embodiments, the isothermal amplification is real-time quantitative isothermal amplification, in which a target nucleic acid is amplified at a constant temperature and the target nucleic acid rate of amplification is monitored by fluorescence, turbidity, or similar measures (e.g., NEAR or LAMP). In some cases, RNA (e.g., mRNA) is isolated from a biological sample and is used as a template to synthesize cDNA by reverse-transcription. cDNA molecules are amplified under isothermal amplification conditions such that the production of amplified target nucleic acid can be detected and quantitated.

In particular embodiments, the isothermal amplification is Loop-Mediated Isothermal Amplification (LAMP). LAMP offers selectivity and employs a polymerase and a set of specially designed primers that recognize distinct sequences in the target nucleic acid (see, e.g., Nixon et al., (2014) Bimolecular Detection and Quantitation, 2:4-10; Schuler et al., (2016) Anal Methods., 8:2750-2755; and Schoepp et al., (2017) Sci. Transl. Med., 9:eaal3693). Unlike PCR, the target nucleic acid is amplified at a constant temperature (e.g., 60-65° C.) using multiple inner and outer primers and a polymerase having strand displacement activity. In some instances, an inner primer pair containing a nucleic acid sequence complementary to a portion of the sense and antisense strands of the target nucleic acid initiate LAMP. Following strand displacement synthesis by the inner primers, strand displacement synthesis primed by an outer primer pair can cause release of a single-stranded amplicon. The single-stranded amplicon may serve as a template for further synthesis primed by a second inner and second outer primer that hybridize to the other end of the target nucleic acid and produce a stem-loop nucleic acid structure. In subsequent LAMP cycling, one inner primer hybridizes to the loop on the product and initiates displacement and target nucleic acid synthesis, yielding the original stem-loop product and a new stem-loop product with a stem twice as long. Additionally, the 3′ terminus of an amplicon loop structure serves as initiation site for self-templating strand synthesis, yielding a hairpin-like amplicon that forms an additional loop structure to prime subsequent rounds of self-templated amplification. The amplification continues with accumulation of many copies of the target nucleic acid. The final products of the LAMP process are stem-loop nucleic acids with concatenated repeats of the target nucleic acid in cauliflower-like structures with multiple loops formed by annealing between alternately inverted repeats of a target nucleic acid sequence in the same strand.

In some embodiments, the isothermal amplification assay comprises a digital reverse-transcription loop-mediated isothermal amplification (dRT-LAMP) reaction for quantifying the target nucleic acid (see, e.g., Khorosheva et al., (2016) Nucleic Acid Research, 44:2 e10). Typically, LAMP assays produce a detectable signal (e.g., fluorescence) during the amplification reaction. In some embodiments, fluorescence can be detected and quantified. Any suitable method for detecting and quantifying florescence can be used. In some instances, a device such as Applied Biosystem's QuantStudio can be used to detect and quantify fluorescence from the isothermal amplification assay.

Any suitable method for detecting amplification of a target nucleic acid in a test sample by quantitative real-time isothermal amplification may be used to practice the present methods. In some embodiments, quantitative real-time isothermal amplification of a target nucleic acid in a test sample is determined by detecting of one or more different (distinct) fluorescent labels attached to nucleotides or nucleotide analogs incorporated during isothermal amplification of the target nucleic acid (e.g., 5-FAM (522 nm), ROX (608 nm), FITC (518 nm) and Nile Red (628 nm). In another embodiment, quantitative real-time isothermal amplification of a target nucleic acid in a test sample can be determined by detection of a single fluorophore species (e.g., ROX (608 nm)) attached to nucleotides or nucleotide analogs incorporated during isothermal amplification of the target nucleic acid. In some embodiments, each fluorophore species used emits a fluorescent signal that is distinct from any other fluorophore species, such that each fluorophore can be readily detected among other fluorophore species present in the assay.

In some embodiments, methods of detecting amplification of a target nucleic acid in a test sample by quantitative real-time isothermal amplification can include using intercalating fluorescent dyes, such as SYTO dyes (SYTO 9 or SYTO 82). In some embodiments, methods of detecting amplification of a target nucleic acid in a test sample by quantitative real-time isothermal amplification can include using unlabeled primers to isothermally amplify the target nucleic acid in the test sample, and a labeled probe (e.g., having a fluorophore) to detect isothermal amplification of the target nucleic acid in the test sample. In some embodiments, unlabeled primers are used to isothermally amplify a target nucleic acid present in the test sample, and a probe is used having a 5-FAM dye label on the 5′ end and a minor groove binder (MGB) and non-fluorescent quencher on the 3′ end to detect isothermal amplification of the target nucleic acid (e.g., TaqMan Gene Expression Assays from ThermoFisher Scientific).

In some embodiments, detecting amplification of the target nucleic acid in the test sample is performed using a one-step, or two-step, quantitative real-time isothermal amplification assay. In a one-step quantitative real-time isothermal amplification assay, reverse transcription is combined with quantitative isothermal amplification to form a single quantitative real-time isothermal amplification assay. A one-step assay reduces the number of hands-on manipulations as well as the total time to process a test sample. A two-step assay comprises a first-step, where reverse transcription is performed, followed by a second-step, where quantitative isothermal amplification is performed. It is within the scope of the skilled artisan to determine whether a one-step or two-step assay should be performed.

In some embodiments, a composite biomarker value is calculated based on the Tt (time to threshold) values or a parameter that measures the rapidity of detected signal rise for each of the tested biomarkers. This may be accomplished by, e.g., establishing standard curves for the isothermal or other amplification of the target nucleic acid (e.g., biomarker) and the reference nucleic acid (e.g., housekeeping gene). The standard curves can be obtained by performing real-time isothermal amplification assays using quantitated calibrator samples with multiple known input concentrations. Appropriate methods are provided in, e.g., PCT Publication No. WO 2020/061217, the entire disclosure of which is herein incorporated by reference.

For example, in some embodiments, to generate a standard curve, quantitated calibrator samples are obtained by performing serial dilutions of a quantitated material. For example, a template is serially diluted in a buffer at 10-fold concentration intervals yielding templates covering a range of concentrations from, e.g., approximately 109 copies/μL to approximately 102 copies/μL. The precise concentration of each calibrator sample can be determined using methods known in the art.

To obtain a standard curve, a real-time amplification assay is performed for each aliquot with a known quantity (e.g., 1 μL) of a respective calibrator sample with a respective concentration of the target nucleic acid. In a real-time amplification assay for each respective calibrator sample, the intensity of the fluorescence emitted by intercalating fluorescent dyes (e.g., dsDNA dyes) or fluorescent labels for the target nucleic acid is measured as a function of time. For example, a plot can be generated of fluorescence intensity as a function of time in a real-time quantitative amplification assay. A dashed line can be used to represent a pre-determined threshold intensity, and the elapsed time from the moment when the amplification is started is the time-to-threshold Tt. A respective time-to-threshold value can be determined from each respective fluorescence curve as a function of time. Thus, time-to-threshold values Ttn, Ttn+1, Ttn+2, etc., are obtained for the different calibrator samples.

For exponential amplifications, the time-to-threshold is linearly proportional to the logarithm (e.g., logarithm to base 10) of the starting copy number (also referred to as template abundance). A scatter plot of data points can be generated from the fluorescence curves. Each data point represents a data pair [Log 10(CopyNumber), Tt](note that CopyNumber refers to starting number of copies of a nucleic acid in an amplification assay). In some embodiments, the data points fall approximately on a straight line. A linear regression is then performed on the data points in the plot to obtain the straight line that best fits the data points with the least amount of total deviations. The result of the linear regression is a straight line represented by the following equation,

$$Tt = m \times {Log}_{10} ( CopyNumber ) + b , \qquad ( 1 )$$

where m is the slope of the line, and b is y-intercept. The slope m represents the efficiency of the isothermal amplification of the target nucleic acid; b represents a time-to-threshold as template copy number approaches zero. The straight line represented by Equation (1) is referred to as the standard curve.

In some embodiments, replicates (e.g., triplicates) of isothermal amplification assays may be run for each sample in order to gain a higher level of confidence in the data. Replicate time-to-threshold values can be averaged, and standard deviations can be calculated.

Once the standard curve is established for a given isothermal amplification assay, the standard curve can be used to convert a time-to-threshold value to a starting copy number for future runs of the amplification assay of unknown starting numbers of copies of the target nucleic acid, using the following equation,

$$CopyNumber = {10}^{\frac{Tt - b}{m}} . \qquad ( 2 )$$

Normally, the data points for low copy numbers or very high copy numbers may fall off of the straight line. The range of copy numbers within which the data points can be represented by the straight line is referred to as the dynamic range of the standard curve. The linear relationship between the time-to-threshold and the logarithmic of copy number represented by the standard curve would be valid only within the dynamic range.

If the amplification efficiencies for a target nucleic acid and a reference nucleic acid are different for a given isothermal amplification assay, it may be necessary to obtain separate standard curves for the target nucleic acid and the reference nucleic acid. Thus, two sets of real-time isothermal amplification assays may be performed, one set for establishing the standard curve for the target nucleic acid, the other set for establishing the standard curve for the reference nucleic acid. In cases where multiple target nucleic acids are considered (e.g., for a panel of seven biomarkers as described herein), a standard curve for each target nucleic acid may be obtained.

In some embodiments, the standard curves are generated prior to obtaining a test sample. That is, the standard curves are not generated on-board with the quantitative isothermal amplification of the test sample. Such standard curves may be referred to as off-board standard curves. Off-board standard curves may be used for estimating relative abundance values (i.e., expression levels). For example, for a test sample of unknown input concentration of a target nucleic acid, a first real-time amplification assay is performed for a first aliquot of the test sample to obtain a first time-to-threshold value with respect to the target nucleic acid. A second real-time isothermal amplification assay is then performed for a second aliquot of the test sample to obtain a second time-to-threshold value with respect to a reference nucleic acid. The first aliquot and the second aliquot contain substantially the same amount of the test sample. The first time-to-threshold value may then be converted into starting number of copies of the target nucleic acid using the standard curve of the target nucleic acid. Similarly, the second time-to-threshold value may be converted into starting number of copies of the reference nucleic acid using the standard curve of the reference nucleic. The starting number of copies of the target nucleic acid is then normalized against that of the reference nucleic acid to obtain a relative abundance value (i.e., expression level).

In cases where the amplification efficiencies for a target nucleic acid and a reference nucleic acid have approximately the same value that is known, relative abundance (i.e., expression level) may be obtained directly from time-to-threshold values without using standard curves.

### V. Calculating Composite Biomarker Values or Biomarker Scores

To determine whether the subject is undergoing MI, a calculation is applied to the biomarker expression data from the subject to determine a composite biomarker value or biomarker score, that is indicative of the probability of the subject undergoing MI. In some embodiments, the calculation for the composite biomarker value or biomarker score can be the geometric mean of the normalized, log 2-transformed expression of the up-regulated mRNA minus that of the down-regulated mRNA from the final biomarker gene signature. The composite biomarker values or biomarker scores can be scaled for comparison between datasets and used for receiver operating characteristic (ROC) curve and area under curve (AUC) as performance metrics of the selected biomarkers.

In some embodiments, the calculation for the composite biomarker value or biomarker score can be the geometric mean of the normalized, log 2-transformed expression of the up-regulated mRNA minus that of the down-regulated mRNA from the biomarker gene signature comprising one or more biomarkers (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 90, 95, 100, 200, 300, or 400 biomarkers) listed in Table 2.

In some embodiments, the calculation for the composite biomarker value or biomarker score can be the geometric mean of the normalized, log 2-transformed expression of the up-regulated mRNA minus that of the down-regulated mRNA from the biomarker gene signature comprising one or more pairs of biomarkers (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 pairs of biomarkers) listed in Table 3.

In some embodiments, the calculation for the composite biomarker value or biomarker score can be the geometric mean of the normalized, log 2-transformed expression of the up-regulated mRNA minus that of the down-regulated mRNA from the biomarker gene signature comprising one or more biomarkers (e.g., 1, 2, 3, 4, 5, 6, 7, 8, 9, 10, 11, 12, 13, 14, or 15 biomarkers) selected from the group consisting of SOCS3, OVCA2, MEF2A, PRR33, SCX, AQP9, IPPK, TNFSF8, CD163, MEF2B, HINT2, ATP1B1, ZCCHC18, CXorf65, and IMP3.

In some embodiments, the calculation for the composite biomarker value or biomarker score can be the geometric mean of the normalized, log 2-transformed expression of the up-regulated mRNA minus that of the down-regulated mRNA from the biomarker gene signature comprising one or more biomarkers (e.g., 1, 2, 3, 4, 5, 6, 7, or 8 biomarkers) selected from the group consisting of SOCS3, SCX, AQP9, IPPK, CD163, ATP1B1, ZCCHC18, and CXorf65.

In some embodiments, the calculation for the composite biomarker value or biomarker score can be the geometric mean of the normalized, log 2-transformed expression of the up-regulated mRNA minus that of the down-regulated mRNA from the biomarker gene signature comprising one or more biomarkers (e.g., 1, 2, 3, 4, 5, 6, or 7 biomarkers) selected from the group consisting of OVCA2, MEF2A, PRR33, TNFSF8, MEF2B, HINT2, and IMP3.

In some embodiments, the composite biomarker value or biomarker score can be calculated, e.g., by taking the sum, product, or quotient of the gene expression levels of the biomarkers, taken in terms of their absolute levels or their relative levels as compared to control genes, e.g., housekeeping genes, or by inputting them into a linear or nonlinear algorithm that incorporates at least the measured expression levels into an interpretable value.

In some embodiments, a threshold or cut-off value is suitably determined, and is optionally a predetermined value. In particular embodiments, the threshold value is predetermined in the sense that it is fixed, for example, based on previous experience with the assay and/or a population of subjects with a given outcome or outcomes, e.g., with a population of 50, 100, 150, 200, 250, 300, 350, 400, 450, 500, or more subjects who underwent or who did not undergo MI. Alternatively, the predetermined value can also indicate that the method of arriving at the threshold is predetermined or fixed even if the particular value varies among assays or can even be determined for every assay run.

For the statistical analyses described herein, e.g., for the selection of biomarkers to be included in the calculation of a composite biomarker value or in the calculation of a probability or likelihood of a subject undergoing MI, as well as for diagnostic or therapeutic assessments made in view of a given composite biomarker value, other relevant information can also be considered, such as clinical data regarding the symptoms presented by each individual. This can include demographic information such as age, race, and sex; information regarding a presence, absence, degree, stage, severity or progression of a condition, phenotypic information, such as details of phenotypic traits, genetic or genetically regulated information, amino acid or nucleotide related genomics information, results of other tests including imaging, biochemical and hematological assays, other physiological scores, or the like.

As described above, the abundance values (i.e., expression levels) for the individual biomarker genes in the biological sample can be combined using a mathematical formula or a machine learning or other algorithm to produce a single composite biomarker value that can indicate the likelihood of a subject undergoing MI. In these embodiments, the produced value carries more predictive power than any individual gene level alone.

In some embodiments, types of algorithms for integrating multiple biomarkers into a single composite biomarker value or biomarker score may include, but are not limited to, a difference of geometric means, a difference of arithmetic means, a difference of sums, a simple sum, and the like. In some embodiments, a composite biomarker value may be estimated based on the relative abundance values (i.e., expression levels) of multiple biomarkers using machine-learning models, such as a regression model, a tree-based machine-learning model, a support vector machine (SVM) model, an artificial neural network (ANN) model, or the like.

Biomarker data may also be analyzed by a variety of methods to determine the statistical significance of differences in observed levels of biomarkers between test and reference expression profiles in order to evaluate the probability of a subject undergoing MI. In certain embodiments, patient data is analyzed by one or more methods including, but not limited to, multivariate linear discriminant analysis (LDA), receiver operating characteristic (ROC) analysis, principal component analysis (PCA), ensemble data mining methods, significance analysis of microarrays (SAM), cell specific significance analysis of microarrays (csSAM), spanning-tree progression analysis of density-normalized events (SPADE), and multi-dimensional protein identification technology (MUDPIT) analysis. (See, e.g., Hilbe (2009) Logistic Regression Models, Chapman & Hall/CRC Press; McLachlan (2004) Discriminant Analysis and Statistical Pattern Recognition. Wiley Interscience; Zweig et al. (1993) Clin. Chem. 39:561-577; Pepe (2003) The statistical evaluation of medical tests for classification and prediction, New York, N.Y.: Oxford; Sing et al. (2005) Bioinformatics 21:3940-3941; Tusher et al. (2001) Proc. Natl. Acad. Sci. U.S.A. 98:5116-5121; Oza (2006) Ensemble data mining, NASA Ames Research Center, Moffett Field, Calif., USA; English et al. (2009) J. Biomed. Inform. 42(2):287-295; Zhang (2007) Bioinformatics 8: 230; Shen-Orr et al. (2010) Journal of Immunology 184:144-130; Qiu et al. (2011) Nat. Biotechnol. 29(10):886-891; Ru et al. (2006) J. Chromatogr. A. 1111(2):166-174, Jolliffe Principal Component Analysis (Springer Series in Statistics, 2.sup.nd edition, Springer, N Y, 2002), Koren et al. (2004) IEEE Trans Vis Comput Graph 10:459-470; herein incorporated by reference in their entireties.)

It is not necessary that all of the biomarkers are elevated or depressed relative to control levels in a biological sample from a given subject to give rise to a determination on whether the subject is undergoing MI. For example, for a given biomarker level there can be some overlap between individuals falling into different probability categories. However, collectively the combined levels for all of the biomarker genes included in the assay will give rise to a value that, when compared to a threshold value, e.g., a threshold value derived from at least 50, 100, 150, 200, 250, 300, 350, 400, 500 or more subjects who have undergone MI, allows a determination concerning whether the subject is undergoing MI. For example, for a determination that a subject has a high likelihood of undergoing MI, the threshold value could be such that at across a population of at least 100 subjects who underwent MI and 100 subjects who did not undergo MI, at least 90% of the subjects who underwent MI are above the threshold. In another example, for a determination that a subject has a low likelihood of undergoing MI, the threshold value could be such that at across a population of at least 100 subjects who underwent MI and 100 subjects who did not undergo MI, at least 90% of the subjects who did not undergo MI are below the threshold. It will be appreciated that in any given assay there can be more than one threshold, e.g., a threshold in one direction that indicates that a subject is undergoing MI, and a threshold in the other direction that indicates that a subject is not undergoing MI.

As used herein, the terms “probability,” and “risk” with respect to a given outcome refer to conditional probability that subjects with a particular value actually have the condition (e.g., undergoing MI) based on a given mathematical model. An increased probability or risk for example can be relative or absolute and can be expressed qualitatively or quantitatively. For instance, an increased risk can be expressed as simply determining the subject's value and placing the test subject in an “increased risk” category, based upon previous population studies. Alternatively, a numerical expression of the test subject's increased risk can be determined based upon an analysis of the composite biomarker value.

In some embodiments, likelihood is assessed by comparing the level of a composite biomarker value or biomarker score to one or more preselected or threshold levels. Threshold values can be selected that provide an acceptable ability to predict the likelihood of a subject undergoing MI. In illustrative examples, receiver operating characteristic (ROC) curves are calculated by plotting the value of a composite biomarker value in two populations in which a first population has a first condition (e.g., undergoing MI) and a second population has a second condition (e.g., not undergoing MI).

For any particular biomarker, a distribution of biomarker levels for subjects with and without a disease will likely overlap, and some overlap will be present for composite biomarker values as well. Under such conditions, a test does not absolutely distinguish a first condition and a second condition with 100% accuracy, and the area of overlap indicates where the test cannot distinguish the first condition and the second condition. A threshold value is selected, above which (or below which, depending on how a composite biomarker value changes with a specified condition or prognosis) the test is considered to be “positive” and below which the test is considered to be “negative.” The area under the ROC curve (AUC) provides the C-statistic, which is a measure of the probability that the perceived measurement will allow correct identification of a condition (see, e.g., Hanley et al., Radiology 143: 29-36 (1982)).

In some embodiments, a positive likelihood ratio, negative likelihood ratio, odds ratio, and/or AUC or receiver operating characteristic (ROC) values are used as a measure of a method's ability to predict the likelihood of a subject undergoing MI. As used herein, the term “likelihood ratio” is the probability that a given test result would be observed in a subject with a condition or outcome of interest (e.g., undergoing MI) divided by the probability that that same result would be observed in a patient without the condition or outcome of interest (e.g., not undergoing MI). Thus, a positive likelihood ratio is the probability of a positive result observed in subjects with the specified condition or outcome divided by the probability of a positive results in subjects without the specified condition or outcome. A negative likelihood ratio is the probability of a negative result in subjects without the specified condition or outcome divided by the probability of a negative result in subjects with specified condition or outcome.

The term “odds ratio,” as used herein, refers to the ratio of the odds of an event occurring in one group (e.g., not undergoing MI) to the odds of it occurring in another group (e.g., undergoing MI), or to a data-based estimate of that ratio. The term “area under the curve” or “AUC” refers to the area under the curve of a receiver operating characteristic (ROC) curve, both of which are well known in the art. AUC measures are useful for evaluating the accuracy of a classifier across the complete decision threshold range. Classifiers with a greater AUC have a greater capacity to classify unknowns correctly between two or more groups of interest (e.g., subjects undergoing or not undergoing MI), or a low, intermediate, or high probability of undergoing MI). ROC curves are useful for plotting the performance of a particular feature (e.g., any of the biomarker expression levels or composite biomarker values described herein and/or any item of additional biomedical information) in distinguishing or discriminating between two populations (e.g., subjects undergoing or not undergoing MI). Typically, the feature data across the entire population (e.g., the cases and controls) are sorted in ascending order based on the value of a single feature. Then, for each value for that feature, the true positive and false positive rates for the data are calculated. The sensitivity is determined by counting the number of cases above the value for that feature and then dividing by the total number of cases. The specificity is determined by counting the number of controls below the value for that feature and then dividing by the total number of controls.

Although this refers to scenarios in which a feature is elevated in cases compared to controls, it also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to produce a single value, and this single value can be plotted in a ROC curve. Additionally, any combination of multiple features, in which the combination derives a single output value, can be plotted in a ROC curve. These combinations of features can comprise a test. The ROC curve is the plot of the sensitivity of a test against 1-specificity of the test, where sensitivity is traditionally presented on the vertical axis and 1-specificity is traditionally presented on the horizontal axis. Thus, “AUC ROC values” are equal to the probability that a classifier will rank a randomly chosen positive instance higher than a randomly chosen negative one.

In some embodiments, at least two (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30 or more) biomarker genes are selected to discriminate between subjects with a first condition or outcome and subjects with a second condition or outcome with at least about 70%, 75%, 80%, 85%, 90%, 95% accuracy or having a C-statistic of at least about 0.70, 0.75, 0.80, 0.85, 0.90, 0.95.

In the case of a positive likelihood ratio, a value of 1 indicates that a positive result is equally likely among subjects in both the “condition” and “control” groups (e.g., in subjects that are undergoing or not undergoing MI); a value greater than 1 indicates that a positive result is more likely in the condition group (e.g., in subjects undergoing MI); and a value less than 1 indicates that a positive result is more likely in the control group (e.g., in subjects not undergoing MI). In this context, “condition” is meant to refer to a group having one characteristic (e.g., undergoing MI) and “control” group lacking the same characteristic (e.g., not undergoing MI). In the case of a negative likelihood ratio, a value of 1 indicates that a negative result is equally likely among subjects in both the “condition” and “control” groups; a value greater than 1 indicates that a negative result is more likely in the “condition” group; and a value less than 1 indicates that a negative result is more likely in the “control” group.

In the case of an odds ratio, a value of 1 indicates that a positive result is equally likely among subjects in both the “condition” and “control” groups; a value greater than 1 indicates that a positive result is more likely in the “condition” group; and a value less than 1 indicates that a positive result is more likely in the “control” group. In the case of an AUC ROC value, this is computed by numerical integration of the ROC curve. The range of this value can be 0.5 to 1.0. A value of 0.5 indicates that a classifier (e.g., a biomarker level) cannot discriminate between cases and controls (e.g., undergoing MI vs not undergoing MI), while 1.0 indicates perfect diagnostic accuracy. In certain embodiments, biomarker gene levels and/or composite biomarker values are selected to exhibit a positive or negative likelihood ratio of at least about 1.5 or more or about 0.67 or less, at least about 2 or more or about 0.5 or less, at least about 5 or more or about 0.2 or less, at least about 10 or more or about 0.1 or less, or at least about 20 or more or about 0.05 or less.

In certain embodiments, the biomarker gene levels and/or composite biomarker values are selected to exhibit an odds ratio of at least about 2 or more or about 0.5 or less, at least about 3 or more or about 0.33 or less, at least about 4 or more or about 0.25 or less, at least about 5 or more or about 0.2 or less, or at least about 10 or more or about 0.1 or less. In certain embodiments, biomarker gene levels and/or composite biomarker values are selected to exhibit an AUC ROC value of greater than 0.5, preferably at least 0.6, more preferably 0.7, still more preferably at least 0.8, even more preferably at least 0.9, and most preferably at least 0.95.

In some cases, multiple thresholds can be determined in so-called “tertile,” “quartile,” or “quintile” analyses. In these methods, the “diseased” and “control groups” (or “high risk” and “low risk”) groups are considered together as a single population, and are divided into 3, 4, or 5 (or more) “bins” having equal numbers of individuals. The boundary between two of these “bins” can be considered “thresholds.” A risk (of a particular diagnosis or prognosis for example) can be assigned based on which “bin” a test subject falls into. In some embodiments of the present methods, subjects are assigned to one of three bins, i.e. “low”, “intermediate”, or “high”, referring to the probability of undergoing MI based on the composite biomarker value obtained using the present methods. For example, subjects can be classified according to the estimated probability of undergoing MI into 3 bins: low likelihood (bin 1), intermediate (bin 2), and high-likelihood (bin 3). The bins are defined, e.g., such that the likelihood ratios are <0.15 in bin 1, from 0.15 to 5 in bin 2, and >5 in bin 3.

The phrases “assessing the likelihood” and “determining the likelihood,” as used herein, refer to methods by which the skilled artisan can predict the presence or absence of a condition (e.g., undergoing MI) in a patient. The skilled artisan will understand that this phrase includes within its scope an increased probability that a condition is present or absent in a patient; that is, that a condition is more likely to be present or absent in a subject. For example, the probability that an individual identified as having a specified condition actually has the condition can be expressed as a “positive predictive value” or “PPV.” Positive predictive value can be calculated as the number of true positives divided by the sum of the true positives and false positives. PPV is determined by the characteristics of the predictive methods of the present methods as well as the prevalence of the condition in the population analyzed. The statistical algorithms can be selected such that the positive predictive value in a population having a condition prevalence is in the range of 70% to 99% and can be, for example, at least 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In other examples, the probability that an individual identified as not having a specified condition or outcome actually does not have that condition can be expressed as a “negative predictive value” or “NPV.” Negative predictive value can be calculated as the number of true negatives divided by the sum of the true negatives and false negatives. Negative predictive value is determined by the characteristics of the diagnostic or prognostic method, system, or code as well as the prevalence of the disease in the population analyzed. The statistical methods and models can be selected such that the negative predictive value in a population having a condition prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, a subject is determined to have a significant probability of having or not having a specified condition or outcome. By “significant probability” is meant that the subject has a reasonable probability (0.6, 0.7, 0.8, 0.9 or more) of having, or not having, a specified condition or outcome.

In some embodiments, the composite biomarker value is combined with one or more clinical parameters. For example, a formula is used to combine (i) either the individual gene expression values or the output from a classifier that uses the gene expression values, with (ii) the one or more clinical parameter-based values or data, to generate (iii) a new value that is useful to the clinician.

### VI. Treatment Decisions

The methods described herein may be used to determine whether the subject is undergoing MI, or can otherwise be used to characterize a sample, in order to correlate outputs of models used for the characterization with a state of MI. The likelihood of a subject undergoing MI can be used to make decisions about further medical care. In some embodiments, a determination of a high probability of a subject undergoing MI can indicate that the subject is a candidate for additional cardiovascular diagnostic testings. Examples of additional cardiovascular testings can include, but are not limited to, angiography (e.g., non-invasive computed tomography (CT) angiography or invasive coronary angiography (ICA)), myocardial perfusion imaging, echocardiography, and magnetic resonance imaging.

In some embodiments, a determination of a high probability of a subject undergoing MI can indicate that the subject is a candidate for further therapeutic interventions. Examples of therapeutic interventions can include, but are not limited to, administration of a pharmaceutical compound, an interventional procedure, or both. Examples of pharmaceutical compounds that can be administered to a subject determined to have a high likelihood of undergoing MI include, but are not limited to, an anticoagulant, an antiplatelet, a beta-blocker, a nitrate, a statin, an angiotensin-converting-enzyme (ACE) inhibitor, and an angiotensin receptor blocker (ARBs). In some embodiments, a subject determined to have a high likelihood of undergoing MI can further receive an interventional procedure, such as revascularization (e.g., a percutaneous coronary intervention (PCI) or a coronary artery bypass graft (CABG)).

In some embodiments, a determination of a low probability of a subject undergoing MI can indicate that the subject is a candidate for further evaluation by a physician for other conditions. For example, a subject with a low probability for undergoing MI can be evaluated by a physician for conditions such as anxiety disorder, aortic dissection, aortic stenosis, asthma, esophagitis, heart failure, gastroenteritis, left ventricular embolism, musculoskeletal disorder, myocarditis, pericarditis, pneumonia, pulmonary embolism, and unstable angina.

In either case, the likelihood of a subject undergoing MI can be used to inform decision making, for example, regarding whether to admit the subject to the medical facility (e.g., a clinic or emergency department) or to release the subject. In some embodiments, regardless of the likelihood of a subject undergoing MI, the subject is likely to receive medical care for the conditions or symptoms that the subject presents, e.g., at the time of entering the clinic or emergency department, either in conjunction with further diagnostic testings or not. As used herein, “medical care” comprises any action taken with respect to the treatment of the subject, whether in an emergency room, urgent care context, another clinical facility or context, or at home, in order to alleviate, eliminate, slow the progression of, or in any way improve any aspect or symptom, including, but not limited to, administering a therapeutic drug, performing surgery, and assisting with symptom management.

### VII. Examples: A Multi-mRNA Signature in Blood Robustly Distinguishes Patients with Myocardial Infarction

### 1. Summary

Transcriptomic data of blood samples from individuals with MI and controls from risk population with CAD or stable/unstable angina were analyzed using multi-cohort analysis of 4 datasets that are clinically meaningful. This analysis allowed for the identification 480 mRNAs that can robustly distinguish MI samples from these control samples with a good accuracy suitable for the clinical setting. The subsets of the 480 identified mRNA biomarkers can be used in developing a new diagnostic test on an established assay system. Such a test, when deployed in clinics and ED, can enable clinicians to make better decisions and boost the current gold standard of triaging MI.

### 2. Datasets

The gene expression omnibus (GEO) and ArrayExpress were surveyed for datasets with transcriptomic data (Homo Sapiens) from either whole blood or peripheral blood mononuclear cell (PBMCs) relevant to cardiovascular diseases, specifically ischemia. For inclusion criteria, datasets that had genome-wide blood-based transcriptomic data and closely mimicked the clinical settings were selected. All datasets were manually curated and 4 were identified. These datasets included both MI samples collected at admission, that can be used as cases, and coronary artery disease (CAD), stable angina (SA), or unstable angina (UA) samples, that can be used as controls against which MI would be discriminated. There were 8 additional datasets that either had MI but used healthy individuals as controls or had MI samples from a later or unknown time point. To mimic the clinical situations better, these 8 datasets were excluded. With the selected 4 datasets of clinical usefulness, there were 80 controls (CAD, SA, or UA) and 193 MI samples (Table 1). Note that in GSE59867, 39 redundant samples were identified empirically based on their transcriptomic profiles that were already included in GSE62646 (28 cases and 11 controls) and thus removed them from the larger set, GSE59867 (instead of original dataset with 111 cases and 46 controls, 83 cases and 35 controls without the confirmed redundant profiles were used). Note that MI samples were from either ST-elevated myocardial infarction (STEMI) or non-ST-elevated myocardial infarction (NSTEMI) events.

TABLE 1
Datasets used for this multi-cohort analysis

| | | | Control | | | |
|---|---|---|---|---|---|---|
| Study | Control | MI | Phenotype | Platform | Year | PMID |
| GSE123342 | 22 | 65 | CAD | GPL17586 | 2019 | 31756302; |
| | | | | | | 34466750 |
| GSE62646 | 14 | 28 | CAD | GPL6244 | 2014 | 23185530 |
| GSE59867 | 35 | 83 | CAD | GPL6244 | 2015 | 25984239 |
| GSE60993 | 9 | 17 | UA | GPL6884 | 2015 | 26025919 |
| Total | 80 | 193 | | | | |

### 3. Methods

Multi-cohort analysis: The four transcriptomic datasets were downloaded from GEO together with their phenotypical data. A well-established multi-cohort analysis6 was performed on these 4 datasets using the Metalntegrator package (v2.1.1) in R. Briefly, effect size (ES) was calculated for each gene within a study between cases (MI samples) and controls (CAD/UA/SA samples) as Hedges' g. The pooled ES across all datasets was computed using DerSimonian & Laird random-effects model. After summarizing the effect size, p-values across all mRNAs were corrected for multiple testing based on Benjamini-Hochberg false discovery rate (FDR). Fisher's sum of logs method was used for combining p-values across studies. Log-sum of p-values that each gene is over- or under-regulated was computed along with corresponding p-values. Again, Benjamini-Hochberg method was used to correct for multiple testing across all mRNAs. Leave one study out (LOSO) analysis was performed by removing one dataset at a time in the discovery.

Identification of signature mRNAs: An effect size (ES) threshold of 0.6 or ≤−0.6 in conjunction with FDR s 0.05 were used to filter differentially expressed mRNAs in the multi-cohort analysis for over-expressed or under-expressed genes respectively. This threshold was empirically chosen to correspond to 80% power for moderate heterogeneity.

Definition of composite biomarker value or biomarker score for the classifier: A classifier value of a sample was evaluated as the geometric mean of the normalized, log 2-transformed expression of the up-regulated mRNA minus that of the down-regulated mRNA from the final gene signature. The values were scaled for comparison between datasets and used for receiver operating characteristic (ROC) curve and area under curve (AUC) as performance metrics of the selected biomarkers.

Down-selection of parsimonious mRNAs: Additionally, from the final biomarker signature, a smaller mRNA set with a similarly robust or optimized performance was identified using a greedy forward search algorithm. Briefly, starting with a list of gene signature, a score, ground truth, and a stopping threshold (0.1), the forward search computes the score for each gene individually and chooses the gene with the highest weighted AUROC across data sets. In subsequent iterations, each of the remaining genes are added to the model one at a time, whereby the gene which provides the greatest increase in weighted AUROC is retained. Once the iterative increase in weighted AUROC falls below the stopping threshold (i.e., the addition of any gene from the list no longer increases the total weighted AUROC by more than the threshold), the forward search terminates, resulting in the final gene list. We defined the weighted AUROC as the sum of each dataset's AUROC multiplied by its number of samples.

### 4. Results

Selection of mRNA biomarker signature: Differential expression was assessed at an ES threshold of 0.6 (over-expressed) or ≤−0.6 (under-expressed) for MI group versus control group in conjunction with FDR s 0.05 for the multi-cohort analysis (Table 2). This threshold was empirically chosen as it corresponds to 80% power for moderate heterogeneity. At |≥ESI 0.6 and FDR ≤0.05, we identified 480 differentially expressed mRNAs in 2 or more out of the 4 studies among a total 18,271 genes covered. We decided to use the 480-mRNA list as our biomarker candidate base. Of these mRNAs, 337 were over-expressed and 143 were under-expressed in MI samples as compared to controls. The effect size and all other parameters are given in Table 2 for each of the identified 480 mRNAs.

Table 2. The list of 480 mRNA biomarkers that distinguish M from control samples. These mRNA have an absolute effect size 0.6 and FDR 0.05 and have been observed in ≥2 or more studies out of the 4 studies. Last column indicates the inclusion flag of the 15 parsimonious genes from forward search.

| | | | | effectSize | FS_Gene |
|---|---|---|---|---|---|
| Entrez ID | Gene Symbol | Gene Name | Effect Size | FDR | (0 = No, 1 = Yes) |
| 9021 | SOCS3 | suppressor of cytokine signaling 3 | −1.447 | 1.49E−22 | 1 |
| 9555 | MACROH2A1 | macroH2A.1 histone | 1.33 | 1.04E−20 | 0 |
| 1378 | CR1 | complement C3b/C4b receptor 1 (Knops | 1.31 | 1.17E−20 | 0 |
| | | blood group) | | | |
| 7462 | LAT2 | linker for activation of T cells family | 1.268 | 5.75E−18 | 0 |
| | | member 2 | | | |
| 1545 | CYP1B1 | cytochrome P450 family 1 subfamily B | 1.26 | 7.15E−18 | 0 |
| | | member 1 | | | |
| 51313 | GASK1B | golgi associated kinase 1B | 1.3 | 2.79E−17 | 0 |
| 3588 | IL10RB | interleukin 10 receptor subunit beta | 1.265 | 1.96E−16 | 0 |
| 81622 | UNC93B1 | unc-93 homolog B1, TLR signaling | 1.316 | 5.63E−16 | 0 |
| | | regulator | | | |
| 3570 | IL6R | interleukin 6 receptor | 1.31 | 7.22E−16 | 0 |
| 6774 | STAT3 | signal transducer and activator of | −1.171 | 8.73E−16 | 0 |
| | | transcription 3 | | | |
| 1052 | CEBPD | CCAAT enhancer binding protein delta | 1.227 | 9.82E−16 | 0 |
| 1535 | CYBA | cytochrome b-245 alpha chain | 1.213 | 1.99E−15 | 0 |
| 1845 | DUSP3 | dual specificity phosphatase 3 | 1.207 | 3.00E−15 | 0 |
| 54502 | RBM47 | RNA binding motif protein 47 | 1.14 | 4.33E−15 | 0 |
| 10226 | PLIN3 | perilipin 3 | 1.079 | 6.55E−15 | 0 |
| 604 | BCL6 | BCL6 transcription repressor | 1.233 | 2.06E−14 | 0 |
| 6948 | TCN2 | transcobalamin 2 | 1.228 | 2.66E−14 | 0 |
| 79660 | PPP1R3B | protein phosphatase 1 regulatory subunit | 1.216 | 5.03E−14 | 0 |
| | | 3B | | | |
| 6556 | SLC11A1 | solute carrier family 11 member 1 | 1.097 | 5.11E−14 | 0 |
| 353514 | LILRA5 | leukocyte immunoglobulin like receptor | 1.144 | 5.42E−14 | 0 |
| | | A5 | | | |
| 2212 | FCGR2A | Fc fragment of IgG receptor IIa | 1.208 | 5.83E−14 | 0 |
| 51363 | CHST15 | carbohydrate sulfotransferase 15 | 1.205 | 6.68E−14 | 0 |
| 1089 | CEACAM4 | CEA cell adhesion molecule 4 | 1.202 | 7.34E−14 | 0 |
| 22918 | CD93 | CD93 molecule | 1.077 | 8.98E−14 | 0 |
| 9489 | PGS1 | phosphatidylglycerophosphate synthase 1 | 1.061 | 2.67E−13 | 0 |
| 10043 | TOM1 | target of myb1 membrane trafficking | 0.996 | 6.79E−13 | 0 |
| | | protein | | | |
| 64207 | IRF2BPL | interferon regulatory factor 2 binding | −1.15 | 8.58E−13 | 0 |
| | | protein like | | | |
| 5265 | SERPINA1 | serpin family A member 1 | 1.086 | 9.77E−13 | 0 |
| 7165 | TPD52L2 | TPD52 like 2 | 1.027 | 1.24E−12 | 0 |
| 533 | ATP6V0B | ATPase H+ transporting V0 subunit b | 1.141 | 1.48E−12 | 0 |
| 53826 | FXYD6 | FXYD domain containing ion transport | 1.091 | 2.15E−12 | 0 |
| | | regulator 6 | | | |
| 3985 | LIMK2 | LIM domain kinase 2 | −0.961 | 3.48E−12 | 0 |
| 2790 | GNG10 | G protein subunit gamma 10 | 1.16 | 5.00E−12 | 0 |
| 1593 | CYP27A1 | cytochrome P450 family 27 subfamily A | 0.95 | 9.94E−12 | 0 |
| | | member 1 | | | |
| 3310 | HSPA6 | heat shock protein family A (Hsp70) | 1.096 | 9.97E−12 | 0 |
| | | member 6 | | | |
| 8291 | DYSF | dysferlin | 0.833 | 2.10E−11 | 0 |
| 5329 | PLAUR | plasminogen activator, urokinase receptor | 1.084 | 2.51E−11 | 0 |
| 8569 | MKNK1 | MAPK interacting serine/threonine kinase | 0.969 | 2.51E−11 | 0 |
| | | 1 | | | |
| 10397 | NDRG1 | N-myc downstream regulated 1 | 1.015 | 2.98E−11 | 0 |
| 3274 | HRH2 | histamine receptor H2 | 1.071 | 3.46E−11 | 0 |
| 206358 | SLC36A1 | solute carrier family 36 member 1 | 1.363 | 3.49E−11 | 0 |
| 64748 | PLPPR2 | phospholipid phosphatase related 2 | 1.066 | 3.85E−11 | 0 |
| 3240 | HP | haptoglobin | 1.006 | 3.85E−11 | 0 |
| 53917 | RAB24 | RAB24, member RAS oncogene family | 1.062 | 3.85E−11 | 0 |
| 83999 | KREMEN1 | kringle containing transmembrane protein | 1.063 | 3.85E−11 | 0 |
| | | 1 | | | |
| 2150 | F2RL1 | F2R like trypsin receptor 1 | 1.059 | 4.91E−11 | 0 |
| 23299 | BICD2 | BICD cargo adaptor 2 | 1.054 | 6.11E−11 | 0 |
| 754 | PTTG1IP | PTTG1 interacting protein | 1.056 | 6.60E−11 | 0 |
| 7132 | TNFRSF1A | TNF receptor superfamily member 1A | 0.989 | 8.89E−11 | 0 |
| 100129307 | LOC100129307 | putative UPF0607 protein | 1.042 | 9.33E−11 | 0 |
| | | ENSP00000383144 | | | |
| 1912 | PHC2 | polyhomeotic homolog 2 | 0.977 | 1.86E−10 | 0 |
| 3101 | HK3 | hexokinase 3 | 1.024 | 1.95E−10 | 0 |
| 6799 | SULT1A2 | sulfotransferase family 1A member 2 | 1.025 | 2.04E−10 | 0 |
| 6283 | S100A12 | S100 calcium binding protein A12 | 1.043 | 2.51E−10 | 0 |
| 91056 | AP5B1 | adaptor related protein complex 5 subunit | 1.017 | 2.68E−10 | 0 |
| | | beta 1 | | | |
| 3460 | IFNGR2 | interferon gamma receptor 2 | 1.081 | 2.68E−10 | 0 |
| 4084 | MXD1 | MAX dimerization protein 1 | −1.014 | 3.16E−10 | 0 |
| 11240 | PADI2 | peptidyl arginine deiminase 2 | 1.012 | 3.32E−10 | 0 |
| 409 | ARRB2 | arrestin beta 2 | 0.953 | 4.74E−10 | 0 |
| 9826 | ARHGEF11 | Rho guanine nucleotide exchange factor | 0.907 | 4.94E−10 | 0 |
| | | 11 | | | |
| 6548 | SLC9A1 | solute carrier family 9 member A1 | 1.28 | 6.26E−10 | 0 |
| 10588 | MTHFS | methenyltetrahydrofolate synthetase | 1.04 | 8.89E−10 | 0 |
| 64386 | MMP25 | matrix metallopeptidase 25 | 0.988 | 8.89E−10 | 0 |
| 10956 | OS9 | OS9 endoplasmic reticulum lectin | 0.988 | 8.89E−10 | 0 |
| 2314 | FLII | FLII actin remodeling protein | 0.987 | 8.99E−10 | 0 |
| 126868 | MAB21L3 | mab-21 like 3 | 0.985 | 1.00E−09 | 0 |
| 79689 | STEAP4 | STEAP4 metalloreductase | 0.981 | 1.17E−09 | 0 |
| 115761 | ARL11 | ADP ribosylation factor like GTPase 11 | −0.966 | 1.17E−09 | 0 |
| 102465753 | NA | NA | 0.929 | 1.18E−09 | 0 |
| 3303 | HSPA1A | heat shock protein family A (Hsp70) | 1.078 | 1.18E−09 | 0 |
| | | member 1A | | | |
| 56606 | SLC2A9 | solute carrier family 2 member 9 | −0.957 | 1.23E−09 | 0 |
| 55701 | ARHGEF40 | Rho guanine nucleotide exchange factor | 0.978 | 1.24E−09 | 0 |
| | | 40 | | | |
| 8650 | NUMB | NUMB endocytic adaptor protein | 0.999 | 1.24E−09 | 0 |
| 407008 | MIR223 | microRNA 223 | 1.035 | 1.34E−09 | 0 |
| 3772 | KCNJ15 | potassium inwardly rectifying channel | 1.191 | 1.37E−09 | 0 |
| | | subfamily J member 15 | | | |
| 29992 | PILRA | paired immunoglobin like type 2 receptor | 0.974 | 1.37E−09 | 0 |
| | | alpha | | | |
| 116844 | LRG1 | leucine rich alpha-2-glycoprotein 1 | 1.265 | 1.51E−09 | 0 |
| 146880 | ARHGAP27P1 | Rho GTPase activating protein 27 | 0.872 | 1.51E−09 | 0 |
| | | pseudogene 1 | | | |
| 23276 | KLHL18 | kelch like family member 18 | 1.181 | 1.63E−09 | 0 |
| 5768 | QSOX1 | quiescin sulfhydryl oxidase 1 | 1.179 | 1.89E−09 | 0 |
| 23569 | PADI4 | peptidyl arginine deiminase 4 | −1.009 | 2.11E−09 | 0 |
| 150372 | NFAM1 | NFAT activating protein with ITAM motif 1 | 0.963 | 2.28E−09 | 0 |
| 124641 | OVCA2 | OVCA2 serine hydrolase domain | 0.912 | 2.37E−09 | 1 |
| | | containing | | | |
| 83862 | TMEM120A | transmembrane protein 120A | −0.958 | 2.54E−09 | 0 |
| 2885 | GRB2 | growth factor receptor bound protein 2 | 0.956 | 2.92E−09 | 0 |
| 2180 | ACSL1 | acyl-CoA synthetase long chain family | 0.863 | 2.92E−09 | 0 |
| | | member 1 | | | |
| 5580 | PRKCD | protein kinase C delta | 0.957 | 3.06E−09 | 0 |
| 51380 | CSAD | cysteine sulfinic acid decarboxylase | 0.953 | 3.08E−09 | 0 |
| 54480 | CHPF2 | chondroitin polymerizing factor 2 | 1.19 | 3.17E−09 | 0 |
| 5226 | PGD | phosphogluconate dehydrogenase | 0.719 | 3.33E−09 | 0 |
| 5209 | PFKFB3 | 6-phosphofructo-2-kinase/fructose-2,6- | 1.332 | 3.33E−09 | 0 |
| | | biphosphatase 3 | | | |
| 9846 | GAB2 | GRB2 associated binding protein 2 | −0.676 | 3.33E−09 | 0 |
| 4616 | GADD45B | growth arrest and DNA damage inducible | 0.953 | 3.41E−09 | 0 |
| | | beta | | | |
| 51296 | SLC15A3 | solute carrier family 15 member 3 | 0.95 | 3.41E−09 | 0 |
| 7039 | TGFA | transforming growth factor alpha | 0.898 | 3.93E−09 | 0 |
| 80271 | ITPKC | inositol-trisphosphate 3-kinase C | 0.897 | 3.93E−09 | 0 |
| 1439 | CSF2RB | colony stimulating factor 2 receptor | 0.951 | 4.19E−09 | 0 |
| | | subunit beta | | | |
| 1889 | ECE1 | endothelin converting enzyme 1 | 0.99 | 4.54E−09 | 0 |
| 1241 | LTB4R | leukotriene B4 receptor | 1.281 | 5.33E−09 | 0 |
| 84067 | FHIP1B | FHF complex subunit HOOK interacting | 1.019 | 5.45E−09 | 0 |
| | | protein 1B | | | |
| 79730 | NSUN7 | NOP2/Sun RNA methyltransferase family | 0.847 | 6.16E−09 | 0 |
| | | member 7 | | | |
| 146712 | B3GNTL1 | UDP-GIcNAc: betaGal beta-1,3-N- | 0.935 | 6.22E−09 | 0 |
| | | acetylglucosaminyltransferase like 1 | | | |
| 3614 | IMPDH1 | inosine monophosphate dehydrogenase 1 | 0.936 | 6.36E−09 | 0 |
| 5509 | PPP1R3D | protein phosphatase 1 regulatory subunit | −0.932 | 7.07E−09 | 0 |
| | | 3D | | | |
| 10175 | CNIH1 | cornichon family AMPA receptor auxiliary | 0.879 | 8.58E−09 | 0 |
| | | protein 1 | | | |
| 1379 | CR1L | complement C3b/C4b receptor 1 like | 0.721 | 9.28E−09 | 0 |
| 2355 | FOSL2 | FOS like 2, AP-1 transcription factor | 0.92 | 1.19E−08 | 0 |
| | | subunit | | | |
| 4205 | MEF2A | myocyte enhancer factor 2A | 0.716 | 1.19E−08 | 1 |
| 8778 | SIGLEC5 | sialic acid binding Ig like lectin 5 | 0.921 | 1.24E−08 | 0 |
| 7408 | VASP | vasodilator stimulated phosphoprotein | −0.906 | 1.41E−08 | 0 |
| 23401 | FRAT2 | FRAT regulator of WNT signaling | 1.296 | 1.43E−08 | 0 |
| | | pathway 2 | | | |
| 3927 | LASP1 | LIM and SH3 protein 1 | −0.915 | 1.50E−08 | 0 |
| 118788 | PIK3AP1 | phosphoinositide-3-kinase adaptor protein | 0.913 | 1.50E−08 | 0 |
| | | 1 | | | |
| 11252 | PACSIN2 | protein kinase C and casein kinase | −0.914 | 1.50E−08 | 0 |
| | | substrate in neurons 2 | | | |
| 5210 | PFKFB4 | 6-phosphofructo-2-kinase/fructose-2,6- | 0.912 | 1.56E−08 | 0 |
| | | biphosphatase 4 | | | |
| 8825 | LIN7A | lin-7 homolog A, crumbs cell polarity | 0.757 | 1.71E−08 | 0 |
| | | complex component | | | |
| 2850 | GPR27 | G protein-coupled receptor 27 | 0.861 | 1.76E−08 | 0 |
| 85450 | ITPRIP | inositol 1,4,5-trisphosphate receptor | −0.904 | 2.10E−08 | 0 |
| | | interacting protein | | | |
| 22856 | CHSY1 | chondroitin sulfate synthase 1 | 0.907 | 2.13E−08 | 0 |
| 11057 | ABHD2 | abhydrolase domain containing 2, | 1.153 | 2.19E−08 | 0 |
| | | acylglycerol lipase | | | |
| 55332 | DRAM1 | DNA damage regulated autophagy | 0.899 | 2.61E−08 | 0 |
| | | modulator 1 | | | |
| 2867 | FFAR2 | free fatty acid receptor 2 | 0.928 | 2.62E−08 | 0 |
| 3055 | HCK | HCK proto-oncogene, Src family tyrosine | −0.897 | 2.88E−08 | 0 |
| | | kinase | | | |
| 3267 | AGFG1 | ArfGAP with FG repeats 1 | 0.858 | 3.20E−08 | 0 |
| 53831 | GPR84 | G protein-coupled receptor 84 | −0.719 | 3.39E−08 | 0 |
| 84418 | CYSTM1 | cysteine rich transmembrane module | 0.805 | 3.78E−08 | 0 |
| | | containing 1 | | | |
| 4688 | NCF2 | neutrophil cytosolic factor 2 | −0.889 | 3.79E−08 | 0 |
| 375 | ARF1 | ADP ribosylation factor 1 | 0.888 | 3.92E−08 | 0 |
| 8660 | IRS2 | insulin receptor substrate 2 | 0.947 | 4.08E−08 | 0 |
| 5660 | PSAP | prosaposin | 0.959 | 4.57E−08 | 0 |
| 2358 | FPR2 | formyl peptide receptor 2 | 0.934 | 4.80E−08 | 0 |
| 257106 | ARHGAP30 | Rho GTPase activating protein 30 | 0.838 | 4.87E−08 | 0 |
| 2878 | GPX3 | glutathione peroxidase 3 | −0.883 | 5.16E−08 | 0 |
| 7100 | TLR5 | toll like receptor 5 | 0.801 | 5.21E−08 | 0 |
| 7409 | VAV1 | vav guanine nucleotide exchange factor 1 | 0.925 | 5.63E−08 | 0 |
| 535 | ATP6V0A1 | ATPase H+ transporting V0 subunit a1 | 1.054 | 5.90E−08 | 0 |
| 2771 | GNAI2 | G protein subunit alpha i2 | 1.05 | 6.04E−08 | 0 |
| 375341 | C3orf62 | chromosome 3 open reading frame 62 | 0.831 | 6.04E−08 | 0 |
| 79143 | MBOAT7 | membrane bound O-acyltransferase | 0.875 | 6.27E−08 | 0 |
| | | domain containing 7 | | | |
| 28232 | SLCO3A1 | solute carrier organic anion transporter | 0.874 | 6.56E−08 | 0 |
| | | family member 3A1 | | | |
| 9170 | LPAR2 | lysophosphatidic acid receptor 2 | 0.872 | 6.70E−08 | 0 |
| 10460 | TACC3 | transforming acidic coiled-coil containing | −0.83 | 6.70E−08 | 0 |
| | | protein 3 | | | |
| 6916 | TBXAS1 | thromboxane A synthase 1 | 0.873 | 6.84E−08 | 0 |
| 64115 | VSIR | V-set immunoregulatory receptor | 1.049 | 7.04E−08 | 0 |
| 11021 | RAB35 | RAB35, member RAS oncogene family | −0.873 | 7.04E−08 | 0 |
| 3340 | NDST1 | N-deacetylase and N-sulfotransferase 1 | 0.871 | 7.45E−08 | 0 |
| 1438 | CSF2RA | colony stimulating factor 2 receptor | −0.871 | 7.45E−08 | 0 |
| | | subunit alpha | | | |
| 346653 | FAM71F2 | family with sequence similarity 71 | 1.286 | 7.45E−08 | 0 |
| | | member F2 | | | |
| 54788 | DNAJB12 | DnaJ heat shock protein family (Hsp40) | 0.805 | 7.56E−08 | 0 |
| | | member B12 | | | |
| 57568 | SIPA1L2 | signal induced proliferation associated 1 | −0.921 | 7.69E−08 | 0 |
| | | like 2 | | | |
| 22921 | MSRB2 | methionine sulfoxide reductase B2 | 0.868 | 7.78E−08 | 0 |
| 51292 | GMPR2 | guanosine monophosphate reductase 2 | 0.867 | 7.88E−08 | 0 |
| 1523 | CUX1 | cut like homeobox 1 | −1.221 | 8.46E−08 | 0 |
| 3753 | KCNE1 | potassium voltage-gated channel | −0.881 | 8.50E−08 | 0 |
| | | subfamily E regulatory subunit 1 | | | |
| 6386 | SDCBP | syndecan binding protein | −0.904 | 8.93E−08 | 0 |
| 9658 | ZNF516 | zinc finger protein 516 | 0.817 | 1.02E−07 | 0 |
| 10023 | FRAT1 | FRAT regulator of WNT signaling | −0.861 | 1.13E−07 | 0 |
| | | pathway 1 | | | |
| 1441 | CSF3R | colony stimulating factor 3 receptor | 0.858 | 1.14E−07 | 0 |
| 152195 | NUDT16L2P | nudix hydrolase 16 like 2, pseudogene | 0.668 | 1.22E−07 | 0 |
| 9802 | DAZAP2 | DAZ associated protein 2 | −0.813 | 1.23E−07 | 0 |
| 100529209 | RNASEK- | RNASEK-C17orf49 readthrough | 0.81 | 1.29E−07 | 0 |
| | C17orf49 | | | | |
| 340120 | ANKRD34B | ankyrin repeat domain 34B | 1.107 | 1.52E−07 | 0 |
| 10106 | CTDSP2 | CTD small phosphatase 2 | 0.768 | 1.56E−07 | 0 |
| 2210 | FCGR1B | Fc fragment of IgG receptor Ib | 1.072 | 1.56E−07 | 0 |
| 10970 | CKAP4 | cytoskeleton associated protein 4 | 1.152 | 1.57E−07 | 0 |
| 978 | CDA | cytidine deaminase | −0.852 | 1.57E−07 | 0 |
| 196383 | RILPL2 | Rab interacting lysosomal protein like 2 | 0.848 | 1.60E−07 | 0 |
| 402483 | LINC01000 | long intergenic non-protein coding RNA | 0.803 | 1.71E−07 | 0 |
| | | 1000 | | | |
| 5447 | POR | cytochrome p450 oxidoreductase | 1.205 | 1.85E−07 | 0 |
| 26524 | LATS2 | large tumor suppressor kinase 2 | −1.115 | 2.00E−07 | 0 |
| 9625 | AATK | apoptosis associated tyrosine kinase | 0.8 | 2.07E−07 | 0 |
| 1453 | CSNK1D | casein kinase 1 delta | 1.123 | 2.14E−07 | 0 |
| 56996 | SLC12A9 | solute carrier family 12 member 9 | −0.842 | 2.14E−07 | 0 |
| 3034 | HAL | histidine ammonia-lyase | 0.84 | 2.19E−07 | 0 |
| 8493 | PPM1D | protein phosphatase, Mg2+/Mn2+ | 0.794 | 2.45E−07 | 0 |
| | | dependent 1D | | | |
| 23645 | PPP1R15A | protein phosphatase 1 regulatory subunit | 0.837 | 2.53E−07 | 0 |
| | | 15A | | | |
| 200315 | APOBEC3A | apolipoprotein B mRNA editing enzyme | 0.757 | 2.56E−07 | 0 |
| | | catalytic subunit 3A | | | |
| 102724536 | PRR33 | proline rich 33 | 1.159 | 2.63E−07 | 1 |
| 58472 | SQOR | sulfide quinone oxidoreductase | 1.103 | 2.64E−07 | 0 |
| 2629 | GBA | glucosylceramidase beta | 0.793 | 2.64E−07 | 0 |
| 133 | ADM | adrenomedullin | 0.957 | 2.71E−07 | 0 |
| 4332 | MNDA | myeloid cell nuclear differentiation antigen | −0.834 | 2.85E−07 | 0 |
| 79746 | ECHDC3 | enoyl-CoA hydratase domain containing 3 | 0.831 | 2.94E−07 | 0 |
| 219972 | MPEG1 | macrophage expressed 1 | 1.221 | 3.04E−07 | 0 |
| 11237 | RNF24 | ring finger protein 24 | 0.83 | 3.14E−07 | 0 |
| 8178 | ELL | elongation factor for RNA polymerase II | 1.126 | 3.17E−07 | 0 |
| 7431 | VIM | vimentin | 1.14 | 3.24E−07 | 0 |
| 11027 | LILRA2 | leukocyte immunoglobulin like receptor | −0.878 | 3.32E−07 | 0 |
| | | A2 | | | |
| 642658 | SCX | scleraxis bHLH transcription factor | −0.827 | 3.47E−07 | 1 |
| 55841 | WWC3 | WWC family member 3 | 0.826 | 3.47E−07 | 0 |
| 290 | ANPEP | alanyl aminopeptidase, membrane | −0.827 | 3.55E−07 | 0 |
| 10645 | CAMKK2 | calcium/calmodulin dependent protein | 0.783 | 4.01E−07 | 0 |
| | | kinase kinase 2 | | | |
| 4210 | MEFV | MEFV innate immuity regulator, pyrin | 1.135 | 4.05E−07 | 0 |
| 115727 | RASGRP4 | RAS guanyl releasing protein 4 | −0.965 | 4.18E−07 | 0 |
| 23092 | ARHGAP26 | Rho GTPase activating protein 26 | 0.822 | 4.18E−07 | 0 |
| 1475 | CSTA | cystatin A | −1.199 | 4.67E−07 | 0 |
| 256435 | ST6GALNAC3 | ST6 N-acetylgalactosaminide alpha-2,6- | −0.743 | 4.76E−07 | 0 |
| | | sialyltransferase 3 | | | |
| 7421 | VDR | vitamin D receptor | 0.817 | 5.00E−07 | 0 |
| 9051 | PSTPIP1 | proline-serine-threonine phosphatase | −0.772 | 5.56E−07 | 0 |
| | | interacting protein 1 | | | |
| 19 | ABCA1 | ATP binding cassette subfamily A | 0.814 | 5.56E−07 | 0 |
| | | member 1 | | | |
| 122402 | TDRD9 | tudor domain containing 9 | −0.813 | 5.59E−07 | 0 |
| 58191 | CXCL16 | C-X-C motif chemokine ligand 16 | 0.813 | 6.29E−07 | 0 |
| 80325 | ABTB1 | ankyrin repeat and BTB domain | 1.137 | 6.29E−07 | 0 |
| | | containing 1 | | | |
| 10673 | TNFSF13B | TNF superfamily member 13b | 0.811 | 6.66E−07 | 0 |
| 81788 | NUAK2 | NUAK family kinase 2 | 0.808 | 6.80E−07 | 0 |
| 9019 | MPZL1 | myelin protein zero like 1 | −0.851 | 6.85E−07 | 0 |
| 7305 | TYROBP | transmembrane immune signaling | 1.084 | 7.05E−07 | 0 |
| | | adaptor TYROBP | | | |
| 7056 | THBD | thrombomodulin | 1.088 | 7.18E−07 | 0 |
| 6272 | SORT1 | sortilin 1 | 0.813 | 7.48E−07 | 0 |
| 6667 | SP1 | Sp1 transcription factor | 0.976 | 7.49E−07 | 0 |
| 9114 | ATP6V0D1 | ATPase H+ transporting V0 subunit d1 | 0.843 | 8.05E−07 | 0 |
| 399844 | LINC01002 | long intergenic non-protein coding RNA | 0.761 | 8.53E−07 | 0 |
| | | 1002 | | | |
| 7706 | TRIM25 | tripartite motif containing 25 | −0.896 | 8.63E−07 | 0 |
| 5230 | PGK1 | phosphoglycerate kinase 1 | 1.126 | 8.83E−07 | 0 |
| 55911 | APOBR | apolipoprotein B receptor | 1.049 | 9.20E−07 | 0 |
| 3732 | CD82 | CD82 molecule | 0.919 | 9.41E−07 | 0 |
| 399744 | LINC00999 | long intergenic non-protein coding RNA | 1.081 | 1.00E−06 | 0 |
| | | 999 | | | |
| 3557 | IL1RN | interleukin 1 receptor antagonist | 1.148 | 1.01E−06 | 0 |
| 4215 | MAP3K3 | mitogen-activated protein kinase kinase | 0.796 | 1.01E−06 | 0 |
| | | kinase 3 | | | |
| 1084 | CEACAM3 | CEA cell adhesion molecule 3 | 0.965 | 1.02E−06 | 0 |
| 7086 | TKT | transketolase | −0.844 | 1.04E−06 | 0 |
| 10288 | LILRB2 | leukocyte immunoglobulin like receptor | 0.86 | 1.10E−06 | 0 |
| | | B2 | | | |
| 140885 | SIRPA | signal regulatory protein alpha | −0.754 | 1.10E−06 | 0 |
| 311 | ANXA11 | annexin A11 | 0.795 | 1.12E−06 | 0 |
| 537 | ATP6AP1 | ATPase H+ transporting accessory | 0.794 | 1.20E−06 | 0 |
| | | protein 1 | | | |
| 11031 | RAB31 | RAB31, member RAS oncogene family | 1.273 | 1.20E−06 | 0 |
| 58484 | NLRC4 | NLR family CARD domain containing 4 | 0.75 | 1.21E−06 | 0 |
| 2970 | GTF2IP1 | general transcription factor IIi | 0.788 | 1.40E−06 | 0 |
| | | pseudogene 1 | | | |
| 51279 | C1RL | complement C1r subcomponent like | 0.657 | 1.41E−06 | 0 |
| 156 | GRK2 | G protein-coupled receptor kinase 2 | 0.787 | 1.44E−06 | 0 |
| 6398 | SECTM1 | secreted and transmembrane 1 | 0.787 | 1.56E−06 | 0 |
| 2353 | FOS | Fos proto-oncogene, AP-1 transcription | −0.935 | 1.57E−06 | 0 |
| | | factor subunit | | | |
| 3684 | ITGAM | integrin subunit alpha M | −0.784 | 1.61E−06 | 0 |
| 128646 | SIRPD | signal regulatory protein delta | 0.785 | 1.64E−06 | 0 |
| 57085 | AGTRAP | angiotensin II receptor associated protein | 0.877 | 1.64E−06 | 0 |
| 57567 | ZNF319 | zinc finger protein 319 | 0.829 | 1.69E−06 | 0 |
| 7099 | TLR4 | toll like receptor 4 | 1.052 | 1.70E−06 | 0 |
| 10409 | BASP1 | brain abundant membrane attached | −0.781 | 1.89E−06 | 0 |
| | | signal protein 1 | | | |
| 112616 | CMTM7 | CKLF like MARVEL transmembrane | 0.78 | 1.92E−06 | 0 |
| | | domain containing 7 | | | |
| 5175 | PECAM1 | platelet and endothelial cell adhesion | 1.047 | 2.03E−06 | 0 |
| | | molecule 1 | | | |
| 284759 | SIRPB2 | signal regulatory protein beta 2 | 1.096 | 2.05E−06 | 0 |
| 116092 | DNTTIP1 | deoxynucleotidyltransferase terminal | −0.775 | 2.23E−06 | 0 |
| | | interacting protein 1 | | | |
| 3304 | HSPA1B | heat shock protein family A (Hsp70) | −0.796 | 2.30E−06 | 0 |
| | | member 1B | | | |
| 54682 | MANSC1 | MANSC domain containing 1 | 0.882 | 2.30E−06 | 0 |
| 1843 | DUSP1 | dual specificity phosphatase 1 | 0.774 | 2.30E−06 | 0 |
| 366 | AQP9 | aquaporin 9 | −0.772 | 2.31E−06 | 1 |
| 8772 | FADD | Fas associated via death domain | 0.949 | 2.31E−06 | 0 |
| 2919 | CXCL1 | C-X-C motif chemokine ligand 1 | 1.007 | 2.39E−06 | 0 |
| 60675 | PROK2 | prokineticin 2 | −0.772 | 2.52E−06 | 0 |
| 494127 | NFYCP2 | NFYC pseudogene 2 | 0.768 | 2.74E−06 | 0 |
| 2123 | EVI2A | ecotropic viral integration site 2A | 0.728 | 2.89E−06 | 0 |
| 199675 | MCEMP1 | mast cell expressed membrane protein 1 | 0.928 | 3.03E−06 | 0 |
| 2131 | EXT1 | exostosin glycosyltransferase 1 | −0.76 | 3.03E−06 | 0 |
| 64768 | IPPK | inositol-pentakisphosphate 2-kinase | −0.834 | 3.04E−06 | 1 |
| 122953 | JDP2 | Jun dimerization protein 2 | 1.034 | 3.24E−06 | 0 |
| 9759 | HDAC4 | histone deacetylase 4 | −0.763 | 3.34E−06 | 0 |
| 7030 | TFE3 | transcription factor binding to IGHM | −0.723 | 3.34E−06 | 0 |
| | | enhancer 3 | | | |
| 79623 | GALNT14 | polypeptide N- | −0.85 | 3.36E−06 | 0 |
| | | acetylgalactosaminyltransferase 14 | | | |
| 2357 | FPR1 | formyl peptide receptor 1 | −0.723 | 3.57E−06 | 0 |
| 10695 | CNPY3 | canopy FGF signaling regulator 3 | 0.938 | 3.64E−06 | 0 |
| 9732 | DOCK4 | dedicator of cytokinesis 4 | 0.8 | 3.96E−06 | 0 |
| 27020 | NPTN | neuroplastin | 0.957 | 3.96E−06 | 0 |
| 155061 | ZNF746 | zinc finger protein 746 | 0.847 | 4.04E−06 | 0 |
| 65220 | NADK | NAD kinase | 0.815 | 4.16E−06 | 0 |
| 11326 | VSIG4 | V-set and immunoglobulin domain | 0.846 | 4.17E−06 | 0 |
| | | containing 4 | | | |
| 11024 | LILRA1 | leukocyte immunoglobulin like receptor | 1.012 | 4.22E−06 | 0 |
| | | A1 | | | |
| 2529 | FUT7 | fucosyltransferase 7 | 0.998 | 4.23E−06 | 0 |
| 10602 | CDC42EP3 | CDC42 effector protein 3 | −0.717 | 4.23E−06 | 0 |
| 9545 | RAB3D | RAB3D, member RAS oncogene family | 0.756 | 4.25E−06 | 0 |
| 2153 | F5 | coagulation factor V | 0.908 | 4.45E−06 | 0 |
| 23294 | ANKS1A | ankyrin repeat and sterile alpha motif | −0.754 | 4.45E−06 | 0 |
| | | domain containing 1A | | | |
| 334 | APLP2 | amyloid beta precursor like protein 2 | 1.18 | 4.45E−06 | 0 |
| 10313 | RTN3 | reticulon 3 | 0.714 | 4.45E−06 | 0 |
| 116369 | SLC26A8 | solute carrier family 26 member 8 | −0.713 | 4.72E−06 | 0 |
| 9842 | PLEKHM1 | pleckstrin homology and RUN domain | 0.789 | 4.72E−06 | 0 |
| | | containing M1 | | | |
| 79887 | PLBD1 | phospholipase B domain containing 1 | 0.873 | 5.20E−06 | 0 |
| 2219 | FCN1 | ficolin 1 | −0.75 | 5.20E−06 | 0 |
| 285848 | PNPLA1 | patatin like phospholipase domain | −0.827 | 5.25E−06 | 0 |
| | | containing 1 | | | |
| 23593 | HEBP2 | heme binding protein 2 | −0.711 | 5.54E−06 | 0 |
| 144195 | SLC2A14 | solute carrier family 2 member 14 | 0.9 | 5.61E−06 | 0 |
| 7378 | UPP1 | uridine phosphorylase 1 | 1.1 | 5.91E−06 | 0 |
| 440093 | H3-5 | H3.5 histone | 0.744 | 5.99E−06 | 0 |
| 1398 | CRK | CRK proto-oncogene, adaptor protein | 0.807 | 6.03E−06 | 0 |
| 7439 | BEST1 | bestrophin 1 | −0.942 | 6.45E−06 | 0 |
| 23765 | IL17RA | interleukin 17 receptor A | 0.789 | 6.74E−06 | 0 |
| 9150 | CTDP1 | CTD phosphatase subunit 1 | 1.067 | 6.81E−06 | 0 |
| 7133 | TNFRSF1B | TNF receptor superfamily member 1B | 0.74 | 6.92E−06 | 0 |
| 83706 | FERMT3 | FERM domain containing kindlin 3 | 0.838 | 7.14E−06 | 0 |
| 80896 | NPL | N-acetylneuraminate pyruvate lyase | 0.828 | 7.21E−06 | 0 |
| 84674 | CARD6 | caspase recruitment domain family | 1.028 | 7.21E−06 | 0 |
| | | member 6 | | | |
| 10924 | SMPDL3A | sphingomyelin phosphodiesterase acid | 0.673 | 7.31E−06 | 0 |
| | | like 3A | | | |
| 124935 | SLC43A2 | solute carrier family 43 member 2 | −0.802 | 7.31E−06 | 0 |
| 219931 | TPCN2 | two pore segment channel 2 | −0.737 | 7.69E−06 | 0 |
| 91662 | NLRP12 | NLR family pyrin domain containing 12 | 0.735 | 8.02E−06 | 0 |
| 2870 | GRK6 | G protein-coupled receptor kinase 6 | 0.899 | 8.12E−06 | 0 |
| 240 | ALOX5 | arachidonate 5-lipoxygenase | −0.697 | 8.26E−06 | 0 |
| 254896 | LOC254896 | uncharacterized LOC254896 | −0.775 | 8.85E−06 | 0 |
| 10221 | TRIB1 | tribbles pseudokinase 1 | 0.731 | 9.06E−06 | 0 |
| 3566 | IL4R | interleukin 4 receptor | −0.732 | 9.38E−06 | 0 |
| 10419 | PRMT5 | protein arginine methyltransferase 5 | 0.73 | 9.90E−06 | 0 |
| 1263 | PLK3 | polo like kinase 3 | 0.771 | 1.05E−05 | 0 |
| 55909 | BIN3 | bridging integrator 3 | −0.71 | 1.05E−05 | 0 |
| 400410 | ST20 | suppressor of tumorigenicity 20 | 0.863 | 1.07E−05 | 0 |
| 51108 | METTL9 | methyltransferase like 9 | 1.081 | 1.07E−05 | 0 |
| 55819 | RNF130 | ring finger protein 130 | 1.383 | 1.08E−05 | 0 |
| 51271 | UBAP1 | ubiquitin associated protein 1 | −0.725 | 1.18E−05 | 0 |
| 79627 | OGFRL1 | opioid growth factor receptor like 1 | −0.723 | 1.21E−05 | 0 |
| 112574 | SNX18 | sorting nexin 18 | −0.722 | 1.27E−05 | 0 |
| 11025 | LILRB3 | leukocyte immunoglobulin like receptor | −0.813 | 1.31E−05 | 0 |
| | | B3 | | | |
| 10610 | ST6GALNAC2 | ST6 N-acetylgalactosaminide alpha-2,6- | −0.685 | 1.37E−05 | 0 |
| | | sialyltransferase 2 | | | |
| 944 | TNFSF8 | TNF superfamily member 8 | 0.86 | 1.39E−05 | 1 |
| 8804 | CREG1 | cellular repressor of E1A stimulated | −0.72 | 1.39E−05 | 0 |
| | | genes 1 | | | |
| 340527 | NHSL2 | NHS like 2 | −0.752 | 1.45E−05 | 0 |
| 5595 | MAPK3 | mitogen-activated protein kinase 3 | 0.718 | 1.46E−05 | 0 |
| 2896 | GRN | granulin precursor | 0.879 | 1.50E−05 | 0 |
| 84649 | DGAT2 | diacylglycerol O-acyltransferase 2 | 1.186 | 1.61E−05 | 0 |
| 1955 | MEGF9 | multiple EGF like domains 9 | 0.842 | 1.61E−05 | 0 |
| 9123 | SLC16A3 | solute carrier family 16 member 3 | 0.714 | 1.61E−05 | 0 |
| 100288687 | DUX4 | double homeobox 4 | 0.819 | 1.62E−05 | 0 |
| 3916 | LAMP1 | lysosomal associated membrane protein | 0.713 | 1.72E−05 | 0 |
| | | 1 | | | |
| 2215 | FCGR3B | Fc fragment of IgG receptor IIIb | 0.71 | 1.79E−05 | 0 |
| 100506557 | NA | NA | −0.71 | 1.79E−05 | 0 |
| 171389 | NLRP6 | NLR family pyrin domain containing 6 | −0.71 | 1.82E−05 | 0 |
| 80262 | PHAF1 | phagosome assembly factor 1 | −0.707 | 2.01E−05 | 0 |
| 9189 | ZBED1 | zinc finger BED-type containing 1 | −0.928 | 2.08E−05 | 0 |
| 9332 | CD163 | CD163 molecule | −0.807 | 2.11E−05 | 1 |
| 80183 | RUBCNL | rubicon like autophagy enhancer | −0.849 | 2.11E−05 | 0 |
| 3099 | HK2 | hexokinase 2 | 1.282 | 2.13E−05 | 0 |
| 54899 | PXK | PX domain containing serine/threonine | −0.704 | 2.23E−05 | 0 |
| | | kinase like | | | |
| 144423 | GLT1D1 | glycosyltransferase 1 domain containing 1 | 0.897 | 2.26E−05 | 0 |
| 23001 | WDFY3 | WD repeat and FYVE domain containing | −0.998 | 2.39E−05 | 0 |
| | | 3 | | | |
| 9586 | CREB5 | cAMP responsive element binding protein | 0.702 | 2.45E−05 | 0 |
| | | 5 | | | |
| 3689 | ITGB2 | integrin subunit beta 2 | 1.018 | 2.47E−05 | 0 |
| 100271849 | MEF2B | myocyte enhancer factor 2B | 1.046 | 2.48E−05 | 1 |
| 2752 | GLUL | glutamate-ammonia ligase | −0.667 | 2.50E−05 | 0 |
| 64757 | MTARC1 | mitochondrial amidoxime reducing | 0.961 | 2.50E−05 | 0 |
| | | component 1 | | | |
| 57580 | PREX1 | phosphatidylinositol-3,4,5-trisphosphate | 1.122 | 2.58E−05 | 0 |
| | | dependent Rac exchange factor 1 | | | |
| 100133130 | NA | NA | 1.014 | 2.62E−05 | 0 |
| 7465 | WEE1 | WEE1 G2 checkpoint kinase | −0.829 | 2.62E−05 | 0 |
| 100528032 | KLRC4- | KLRC4-KLRK1 readthrough | 1.138 | 2.65E−05 | 0 |
| | KLRK1 | | | | |
| 552900 | BOLA2 | bolA family member 2 | 0.881 | 2.69E−05 | 0 |
| 400986 | ANKRD36C | ankyrin repeat domain 36C | −0.698 | 2.72E−05 | 0 |
| 64682 | ANAPC1 | anaphase promoting complex subunit 1 | −0.697 | 2.72E−05 | 0 |
| 7840 | ALMS1 | ALMS1 centrosome and basal body | 0.694 | 3.06E−05 | 0 |
| | | associated protein | | | |
| 83988 | NCALD | neurocalcin delta | −0.815 | 3.11E−05 | 0 |
| 23035 | PHLPP2 | PH domain and leucine rich repeat | 1.035 | 3.30E−05 | 0 |
| | | protein phosphatase 2 | | | |
| 6125 | RPL5 | ribosomal protein L5 | 0.958 | 3.44E−05 | 0 |
| 25914 | RTTN | rotatin | −0.69 | 3.60E−05 | 0 |
| 57493 | HEG1 | heart development protein with EGF like | −0.804 | 3.63E−05 | 0 |
| | | domains 1 | | | |
| 23545 | ATP6V0A2 | ATPase H+ transporting V0 subunit a2 | 0.898 | 3.63E−05 | 0 |
| 196294 | IMMP1L | inner mitochondrial membrane peptidase | −0.794 | 3.78E−05 | 0 |
| | | subunit 1 | | | |
| 11278 | KLF12 | Kruppel like factor 12 | 1.18 | 3.99E−05 | 0 |
| 54677 | CROT | carnitine O-octanoyltransferase | 0.825 | 4.49E−05 | 0 |
| 677822 | SNORA40 | small nucleolar RNA, H/ACA box 40 | −1.033 | 4.51E−05 | 0 |
| 23480 | SEC61G | SEC61 translocon subunit gamma | 0.893 | 4.51E−05 | 0 |
| 445347 | TARP | TCR gamma alternate reading frame | −0.681 | 4.60E−05 | 0 |
| | | protein | | | |
| 619568 | SNORA4 | small nucleolar RNA, H/ACA box 4 | 1.148 | 4.86E−05 | 0 |
| 9125 | CNOT9 | CCR4-NOT transcription complex subunit | 0.887 | 5.13E−05 | 0 |
| | | 9 | | | |
| 100532731 | COMMD3- | COMMD3-BMI1 readthrough | 0.791 | 5.16E−05 | 0 |
| | BMI1 | | | | |
| 26031 | OSBPL3 | oxysterol binding protein like 3 | −0.676 | 5.22E−05 | 0 |
| 27229 | TUBGCP4 | tubulin gamma complex associated | 0.989 | 5.45E−05 | 0 |
| | | protein 4 | | | |
| 166785 | MMAA | metabolism of cobalamin associated A | 1.04 | 5.54E−05 | 0 |
| 55752 | SEPTIN11 | septin 11 | 0.779 | 5.71E−05 | 0 |
| 6636 | SNRPF | small nuclear ribonucleoprotein | −0.67 | 6.23E−05 | 0 |
| | | polypeptide F | | | |
| 57713 | SFMBT2 | Scm like with four mbt domains 2 | 0.81 | 6.28E−05 | 0 |
| 3001 | GZMA | granzyme A | −0.851 | 6.37E−05 | 0 |
| 22836 | RHOBTB3 | Rho related BTB domain containing 3 | 0.995 | 6.44E−05 | 0 |
| 55706 | NDC1 | NDC1 transmembrane nucleoporin | −0.666 | 7.07E−05 | 0 |
| 347051 | SLC10A5 | solute carrier family 10 member 5 | 1.1 | 7.31E−05 | 0 |
| 79673 | ZNF329 | zinc finger protein 329 | 1.083 | 7.38E−05 | 0 |
| 222484 | LNX2 | ligand of numb-protein X 2 | 1.253 | 7.38E−05 | 0 |
| 10133 | OPTN | optineurin | 0.664 | 7.44E−05 | 0 |
| 148103 | ZNF599 | zinc finger protein 599 | 0.987 | 7.45E−05 | 0 |
| 8934 | RAB29 | RAB29, member RAS oncogene family | −0.663 | 7.45E−05 | 0 |
| 114044 | MCM3AP- | MCM3AP antisense RNA 1 | 0.99 | 7.52E−05 | 0 |
| | AS1 | | | | |
| 84172 | POLR1B | RNA polymerase I subunit B | 0.928 | 7.70E−05 | 0 |
| 5727 | PTCH1 | patched 1 | 0.661 | 7.98E−05 | 0 |
| 6627 | SNRPA1 | small nuclear ribonucleoprotein | −0.662 | 8.28E−05 | 0 |
| | | polypeptide A′ | | | |
| 54602 | NDFIP2 | Nedd4 family interacting protein 2 | −0.867 | 9.00E−05 | 0 |
| 343413 | FCRL6 | Fc receptor like 6 | 0.911 | 9.06E−05 | 0 |
| 10771 | ZMYND11 | zinc finger MYND-type containing 11 | 0.712 | 9.28E−05 | 0 |
| 64754 | SMYD3 | SET and MYND domain containing 3 | −0.988 | 9.46E−05 | 0 |
| 256957 | HEATR9 | HEAT repeat containing 9 | 0.937 | 9.46E−05 | 0 |
| 919 | CD247 | CD247 molecule | 1.119 | 9.46E−05 | 0 |
| 957 | ENTPD5 | ectonucleoside triphosphate | 0.857 | 9.54E−05 | 0 |
| | | diphosphohydrolase 5 (inactive) | | | |
| 284367 | SIGLEC17P | sialic acid binding Ig like lectin 17, | 1.116 | 9.63E−05 | 0 |
| | | pseudogene | | | |
| 57587 | CFAP97 | cilia and flagella associated protein 97 | −0.906 | 9.67E−05 | 0 |
| 100130428 | NA | NA | −0.699 | 9.82E−05 | 0 |
| 125893 | ZNF816 | zinc finger protein 816 | −0.69 | 0.00010707 | 0 |
| 3458 | IFNG | interferon gamma | 1.159 | 0.00010813 | 0 |
| 51678 | PALS2 | protein associated with LIN7 2, MAGUK | 1.13 | 0.00011179 | 0 |
| | | family member | | | |
| 51361 | HOOK1 | hook microtubule tethering protein 1 | 0.65 | 0.00011325 | 0 |
| 83888 | FGFBP2 | fibroblast growth factor binding protein 2 | −1.409 | 0.0001212 | 0 |
| 79022 | TMEM106C | transmembrane protein 106C | −0.814 | 0.00012399 | 0 |
| 8644 | AKR1C3 | aldo-keto reductase family 1 member C3 | 0.847 | 0.00012994 | 0 |
| 9397 | NMT2 | N-myristoyltransferase 2 | −0.806 | 0.0001355 | 0 |
| 100272147 | CMC4 | C-X9-C motif containing 4 | −0.643 | 0.00013583 | 0 |
| 4306 | NR3C2 | nuclear receptor subfamily 3 group C | −0.678 | 0.0001457 | 0 |
| | | member 2 | | | |
| 54847 | SIDT1 | SID1 transmembrane family member 1 | −0.72 | 0.00015405 | 0 |
| 11126 | CD160 | CD160 molecule | 0.867 | 0.00015973 | 0 |
| 4047 | LSS | lanosterol synthase | 0.854 | 0.00016743 | 0 |
| 3141 | HLCS | holocarboxylase synthetase | 0.838 | 0.00018262 | 0 |
| 5243 | ABCB1 | ATP binding cassette subfamily B | 0.75 | 0.00019197 | 0 |
| | | member 1 | | | |
| 2841 | GPR18 | G protein-coupled receptor 18 | 0.631 | 0.00019317 | 0 |
| 54900 | LAX1 | lymphocyte transmembrane adaptor 1 | 0.823 | 0.00019841 | 0 |
| 55112 | DYNC2I1 | dynein 2 intermediate chain 1 | 1.055 | 0.00019901 | 0 |
| 10424 | PGRMC2 | progesterone receptor membrane | 1.081 | 0.00020168 | 0 |
| | | component 2 | | | |
| 79896 | THNSL1 | threonine synthase like 1 | −0.664 | 0.0002138 | 0 |
| 692075 | SNORD6 | small nucleolar RNA, C/D box 6 | −0.741 | 0.0002138 | 0 |
| 25945 | NECTIN3 | nectin cell adhesion molecule 3 | −0.956 | 0.00021696 | 0 |
| 29081 | METTL5 | methyltransferase 5, N6-adenosine | 0.863 | 0.00021744 | 0 |
| 7976 | FZD3 | frizzled class receptor 3 | 0.823 | 0.00022237 | 0 |
| 57504 | MTA3 | metastasis associated 1 family member 3 | −0.806 | 0.0002282 | 0 |
| 23180 | RFTN1 | raftlin, lipid raft linker 1 | 1.443 | 0.00023587 | 0 |
| 84319 | CMSS1 | cms1 ribosomal small subunit homolog | 0.84 | 0.00024451 | 0 |
| 4285 | MIPEP | mitochondrial intermediate peptidase | 0.888 | 0.00024626 | 0 |
| 5984 | RFC4 | replication factor C subunit 4 | −0.786 | 0.00025095 | 0 |
| 23127 | COLGALT2 | collagen beta(1-O)galactosyltransferase 2 | −0.904 | 0.00026995 | 0 |
| 55552 | ZNF823 | zinc finger protein 823 | 0.89 | 0.00028474 | 0 |
| 2176 | FANCC | FA complementation group C | 1.077 | 0.00029758 | 0 |
| 729020 | RPEL1 | ribulose-5-phosphate-3-epimerase like 1 | 1.24 | 0.00029876 | 0 |
| 6296 | ACSM3 | acyl-CoA synthetase medium chain family | 0.834 | 0.00030057 | 0 |
| | | member 3 | | | |
| 26984 | SEC22A | SEC22 homolog A, vesicle trafficking | −0.76 | 0.00030317 | 0 |
| | | protein | | | |
| 9805 | SCRN1 | secernin 1 | 0.874 | 0.00030828 | 0 |
| 79899 | PRR5L | proline rich 5 like | 1.02 | 0.00035234 | 0 |
| 132299 | OCIAD2 | OCIA domain containing 2 | −0.808 | 0.00035859 | 0 |
| 8910 | SGCE | sarcoglycan epsilon | −0.781 | 0.00039374 | 0 |
| 163255 | ZNF540 | zinc finger protein 540 | 1.005 | 0.0004075 | 0 |
| 92070 | CTBP1- | CTBP1 divergent transcript | −0.903 | 0.00040978 | 0 |
| | DT | | | | |
| 57559 | STAMBPL1 | STAM binding protein like 1 | 0.687 | 0.00042306 | 0 |
| 26040 | SETBP1 | SET binding protein 1 | 0.906 | 0.00043291 | 0 |
| 93594 | TBC1D31 | TBC1 domain family member 31 | −0.639 | 0.00043291 | 0 |
| 266812 | NAP1L5 | nucleosome assembly protein 1 like 5 | 0.953 | 0.00044722 | 0 |
| 147949 | ZNF583 | zinc finger protein 583 | −0.732 | 0.00046077 | 0 |
| 80304 | WDCP | WD repeat and coiled coil containing | 1.104 | 0.00049732 | 0 |
| 246243 | RNASEH1 | ribonuclease H1 | 0.707 | 0.00049831 | 0 |
| 8975 | USP13 | ubiquitin specific peptidase 13 | 0.756 | 0.00059773 | 0 |
| 3568 | IL5RA | interleukin 5 receptor subunit alpha | 0.907 | 0.00060888 | 0 |
| 8814 | CDKL1 | cyclin dependent kinase like 1 | −0.848 | 0.00065891 | 0 |
| 10090 | UST | uronyl 2-sulfotransferase | −0.767 | 0.00066079 | 0 |
| 84681 | HINT2 | histidine triad nucleotide binding protein 2 | 1.059 | 0.00074062 | 1 |
| 11174 | ADAMTS6 | ADAM metallopeptidase with | 1.183 | 0.00075538 | 0 |
| | | thrombospondin type 1 motif 6 | | | |
| 400322 | HERC2P2 | HERC2 pseudogene 2 | 0.89 | 0.00075538 | 0 |
| 641 | BLM | BLM RecQ like helicase | −0.615 | 0.00078893 | 0 |
| 692226 | SNORD96B | small nucleolar RNA, C/D box 96B | 0.869 | 0.0007909 | 0 |
| 692085 | SNORD45C | small nucleolar RNA, C/D box 45C | 0.92 | 0.0007937 | 0 |
| 55876 | GSDMB | gasdermin B | −0.723 | 0.00083599 | 0 |
| 481 | ATP1B1 | ATPase Na+/K+ transporting subunit beta | −0.867 | 7460.00086 | 1 |
| | | 1 | | | |
| 26769 | SNORD81 | small nucleolar RNA, C/D box 81 | −0.788 | 0.0008778 | 0 |
| 60491 | NIF3L1 | NGG1 interacting factor 3 like 1 | 0.811 | 0.00088079 | 0 |
| 100131211 | NEMP2 | nuclear envelope integral membrane | 0.948 | 0.0009118 | 0 |
| | | protein 2 | | | |
| 23086 | EXPH5 | exophilin 5 | 1.052 | 0.00100437 | 0 |
| 11022 | TDRKH | tudor and KH domain containing | 1.105 | 0.00102496 | 0 |
| 83943 | IMMP2L | inner mitochondrial membrane peptidase | 0.845 | 0.00109003 | 0 |
| | | subunit 2 | | | |
| 84816 | RTN4IP1 | reticulon 4 interacting protein 1 | 1.033 | 0.00115705 | 0 |
| 57519 | STARD9 | StAR related lipid transfer domain | 0.862 | 0.00119128 | 0 |
| | | containing 9 | | | |
| 644353 | ZCCHC18 | zinc finger CCHC-type containing 18 | −0.738 | 0.00121039 | 1 |
| 100337591 | SNORA70F | small nucleolar RNA, H/ACA box 70F | 1.079 | 0.00123734 | 0 |
| 84216 | TMEM117 | transmembrane protein 117 | −0.919 | 0.0012795 | 0 |
| 5920 | PLAAT4 | phospholipase A and acyltransferase 4 | 1.117 | 0.00130951 | 0 |
| 55449 | DHRS4- | DHRS4 antisense RNA 1 | −0.965 | 0.00139907 | 0 |
| | AS1 | | | | |
| 140883 | ZNF280B | zinc finger protein 280B | −0.763 | 0.00140289 | 0 |
| 79071 | ELOVL6 | ELOVL fatty acid elongase 6 | −0.982 | 0.00145733 | 0 |
| 692088 | SNORD50B | small nucleolar RNA, C/D box 50B | 1.185 | 0.00147692 | 0 |
| 56922 | MCCC1 | methylcrotonyl-CoA carboxylase subunit | 0.901 | 0.00191711 | 0 |
| | | 1 | | | |
| 9498 | SLC4A8 | solute carrier family 4 member 8 | 0.964 | 0.00196182 | 0 |
| 100499483 | CCDC180 | coiled-coil domain containing 180 | −0.889 | 0.00200615 | 0 |
| 51744 | CD244 | CD244 molecule | 1.176 | 0.00201087 | 0 |
| 10152 | ABI2 | abl interactor 2 | 1.285 | 0.00231976 | 0 |
| 79658 | ARHGAP10 | Rho GTPase activating protein 10 | −1.659 | 0.00297122 | 0 |
| 51021 | MRPS16 | mitochondrial ribosomal protein S16 | −0.84 | 0.003211 | 0 |
| 114614 | MIR155HG | MIR155 host gene | −1.046 | 0.00321741 | 0 |
| 91147 | TMEM67 | transmembrane protein 67 | 0.908 | 0.00439349 | 0 |
| 158830 | CXorf65 | chromosome X open reading frame 65 | −0.934 | 0.00759331 | 1 |
| 57494 | RIMKLB | ribosomal modification protein rimK like | −1.296 | 0.0163032 | 0 |
| | | family member B | | | |
| 55272 | IMP3 | IMP U3 small nucleolar ribonucleoprotein | 0.899 | 0.0270218 | 1 |
| | | 3 | | | |
| 692196 | SNORD76 | small nucleolar RNA, C/D box 76 | 0.815 | 0.03079021 | 0 |
| 50624 | CUZD1 | CUB and zona pellucida like domains 1 | −1.172 | 0.03272361 | 0 |
| 388536 | ZNF790 | zinc finger protein 790 | −1.526 | 0.039121974 | 0 |
| 100134869 | UBE2Q2P2 | ubiquitin conjugating enzyme E2 Q2 | 1.805 | 0.05653744 | 0 |
| | | pseudogene 2 | | | |

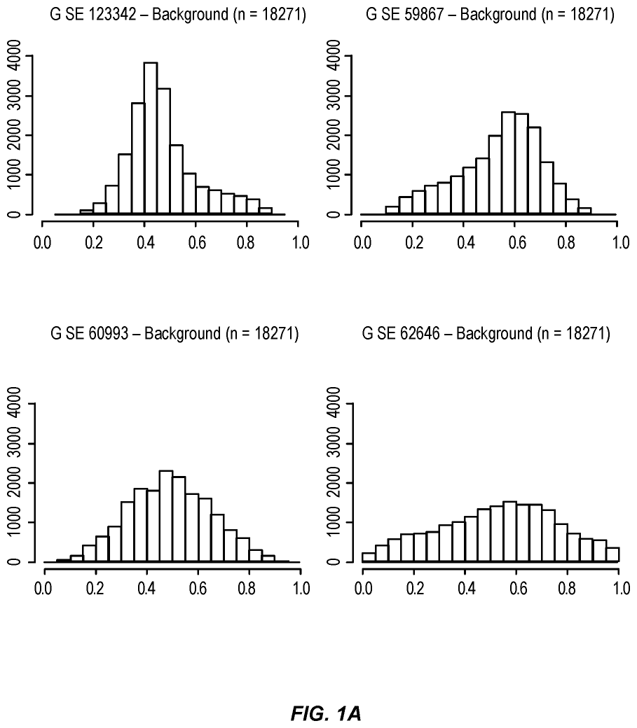

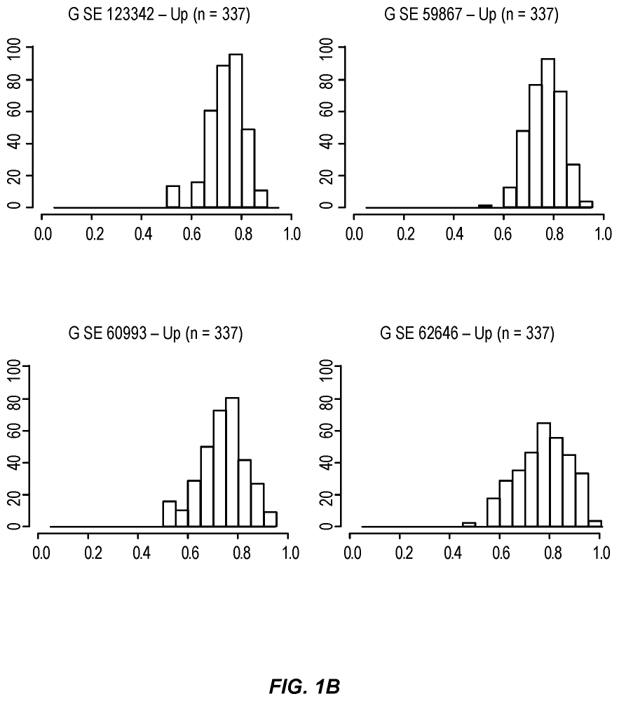

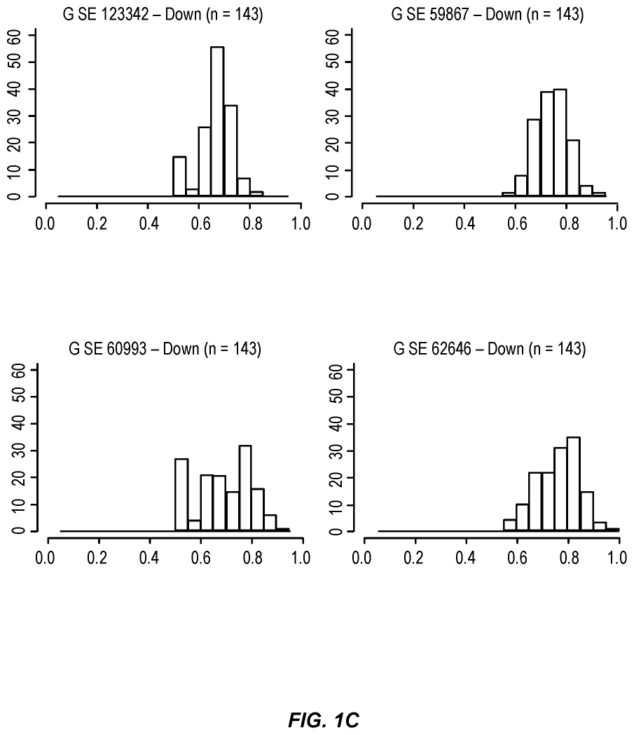

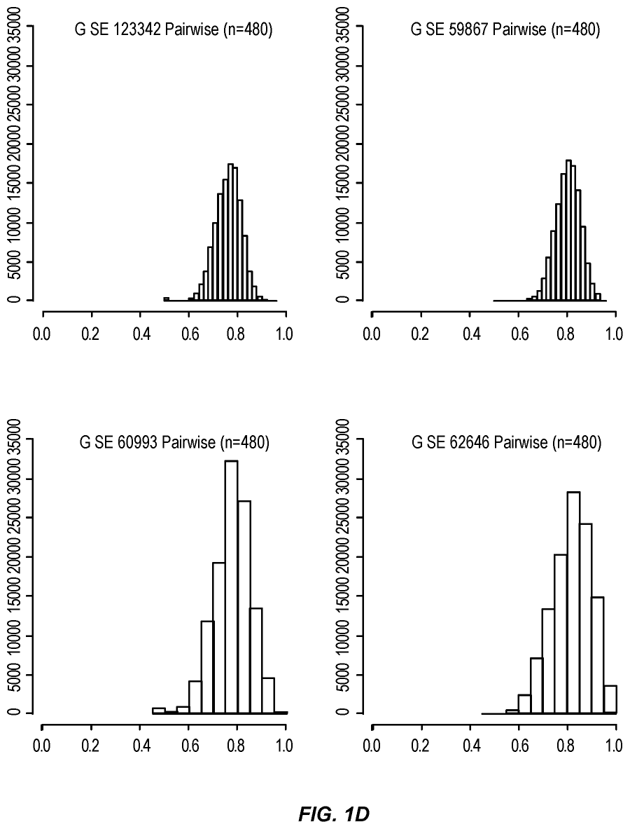

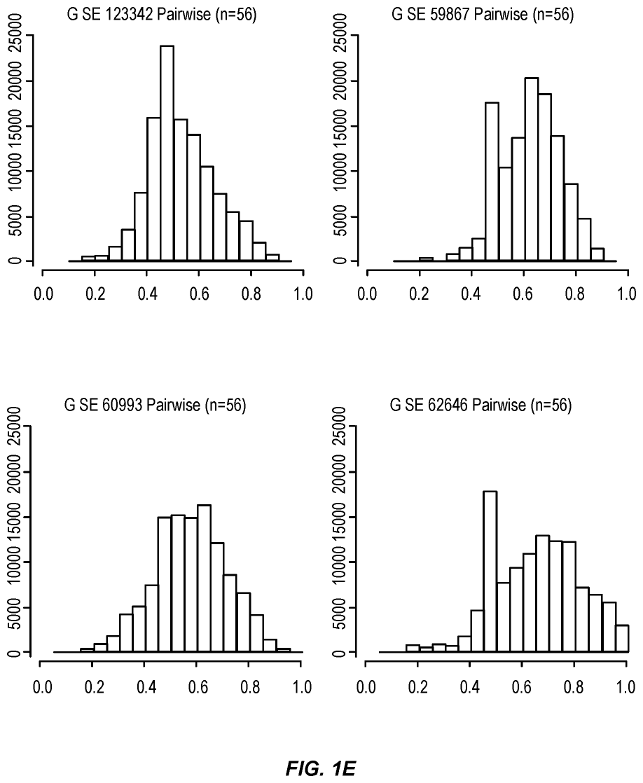

Performance of individual mRNA and two-mRNA combinations: First, AUCs for these ROCs were determined for each of all measured mRNAs (18,271 total) across the 4 studies in order to understand the background characteristics (FIG. 1A) and for each of 480 mRNA that met the cutoff by calculating the single mRNA AUC across all 4 studies (FIGS. 1B and 1C). Additionally, the performance of each of the 114,960 combinations of 2-mRNA pairs formed out of the 480 mRNAs in those 4 datasets was determined (FIG. 1D and Table 3). Note that Table 3 lists 39 top pairs with AUC≥0.85 in all 4 datasets. AUCs for all 114,960 pairs in each of the 4 datasets together with average, standard deviation, and standard error were tabulated. In all 4 datasets, 39 pairs have AUC≥0.85 and 3,735 pairs have AUC 0.80 out of a total 114,960 pairs. On average over 4 datasets, there are 5,602 pairs with AUC≥0.85, 52,229 pairs with AUC≥0.80, and 106,804 pairs with AUC≥0.75. As expected, AUC for single mRNA and 2-mRNA pairs of the selected 480 mRNAs are meaningful in comparison with the background AUCs. Furthermore, as an additional null comparison, AUCs across the 4 studies using pairwise combinations of randomly selected 480 mRNAs were obtained, mimicking a null-hypothesis (FIG. 1E).

Table 3. Performance of top 2-mRNA combinations. We used pairwise gene combinations (114,960 combinations) to evaluate the performance. Top 39 combinations were with AUC 0.85 in all 4 datasets are listed here.

| | | AUC | | | |
|---|---|---|---|---|---|
| Gene 1 | Gene 2 | GSE123342 | GSE59867 | GSE60993 | GSE62646 |
| ADM | TNFSF8 | 0.858 | 0.944 | 0.869 | 0.921 |
| BICD2 | PHLPP2 | 0.851 | 0.854 | 0.895 | 0.867 |
| CR1 | ATP1B1 | 0.85 | 0.868 | 0.915 | 0.878 |
| CR1 | HINT2 | 0.857 | 0.884 | 0.915 | 0.898 |
| CR1 | QSOX1 | 0.858 | 0.885 | 0.948 | 0.888 |
| CR1 | RHOBTB3 | 0.854 | 0.857 | 0.915 | 0.852 |
| CR1 | SLC16A3 | 0.857 | 0.872 | 0.889 | 0.89 |
| CR1 | TNFRSF1B | 0.86 | 0.854 | 0.863 | 0.852 |
| CYP1B1 | ABHD2 | 0.873 | 0.85 | 0.908 | 0.865 |
| CYP1B1 | MXD1 | 0.85 | 0.855 | 0.882 | 0.865 |
| FCN1 | PLAAT4 | 0.855 | 0.856 | 0.85 | 0.898 |
| IL10RB | PHLPP2 | 0.852 | 0.887 | 0.863 | 0.86 |
| IL10RB | STAT3 | 0.852 | 0.874 | 0.85 | 0.872 |
| IL6R | ENTPD5 | 0.866 | 0.856 | 0.882 | 0.875 |
| LILRA5 | HINT2 | 0.854 | 0.883 | 0.85 | 0.957 |
| LILRA5 | TNFSF8 | 0.88 | 0.894 | 0.856 | 0.888 |
| MACROH2A1 | MXD1 | 0.855 | 0.85 | 0.876 | 0.872 |
| MACROH2A1 | NIF3L1 | 0.859 | 0.852 | 0.876 | 0.939 |
| MKNK1 | PHLPP2 | 0.855 | 0.867 | 0.876 | 0.893 |
| MXD1 | OVCA2 | 0.868 | 0.857 | 0.902 | 0.908 |
| MXD1 | QSOX1 | 0.866 | 0.864 | 0.889 | 0.903 |
| PHC2 | APLP2 | 0.853 | 0.856 | 0.856 | 0.852 |
| PPP1R3B | QSOX1 | 0.855 | 0.872 | 0.928 | 0.959 |
| QSOX1 | ACSL1 | 0.852 | 0.882 | 0.895 | 0.867 |
| QSOX1 | AGFG1 | 0.891 | 0.853 | 0.863 | 0.921 |
| QSOX1 | ENTPD5 | 0.871 | 0.893 | 0.908 | 0.941 |
| QSOX1 | MNDA | 0.852 | 0.854 | 0.902 | 0.86 |
| QSOX1 | NDFIP2 | 0.852 | 0.863 | 0.863 | 0.944 |
| QSOX1 | PPP1R3D | 0.867 | 0.854 | 0.889 | 0.974 |
| QSOX1 | SIGLEC17P | 0.91 | 0.869 | 0.85 | 0.931 |
| QSOX1 | SLC2A14 | 0.861 | 0.852 | 0.935 | 0.857 |
| QSOX1 | TMEM106C | 0.876 | 0.853 | 0.915 | 0.931 |
| QSOX1 | TRIM25 | 0.856 | 0.865 | 0.856 | 0.952 |
| QSOX1 | UPP1 | 0.869 | 0.877 | 0.876 | 0.885 |
| SOCS3 | TNFSF8 | 0.855 | 0.93 | 0.974 | 0.967 |
| STAT3 | QSOX1 | 0.859 | 0.852 | 0.915 | 0.972 |
| TCN2 | TNFSF8 | 0.851 | 0.872 | 0.863 | 0.954 |
| TNFSF8 | CREB5 | 0.914 | 0.866 | 0.869 | 0.926 |
| ZNF746 | APLP2 | 0.864 | 0.853 | 0.85 | 0.911 |

Performance of 480 mRNA biomarker set: The geometric mean value calculated based on the description in Methods for the 480 selected mRNAs were significantly higher for MI samples as compared to the control samples in all datasets (FIG. 2A). The corresponding AUROCs based on the value demonstrated the high discriminating power of such a value in differentiating MI samples from controls (FIG. 2B).

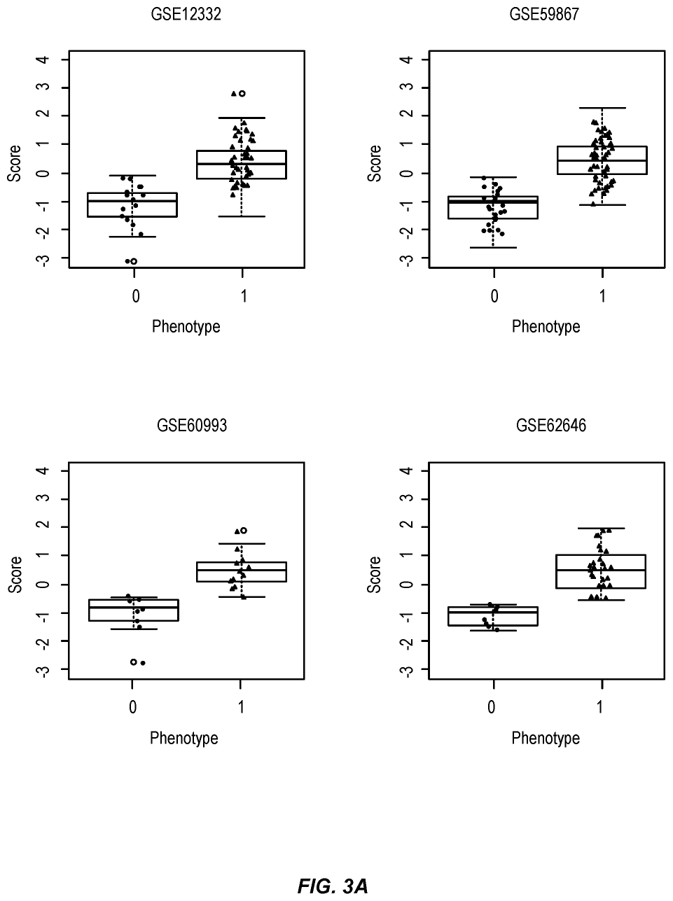

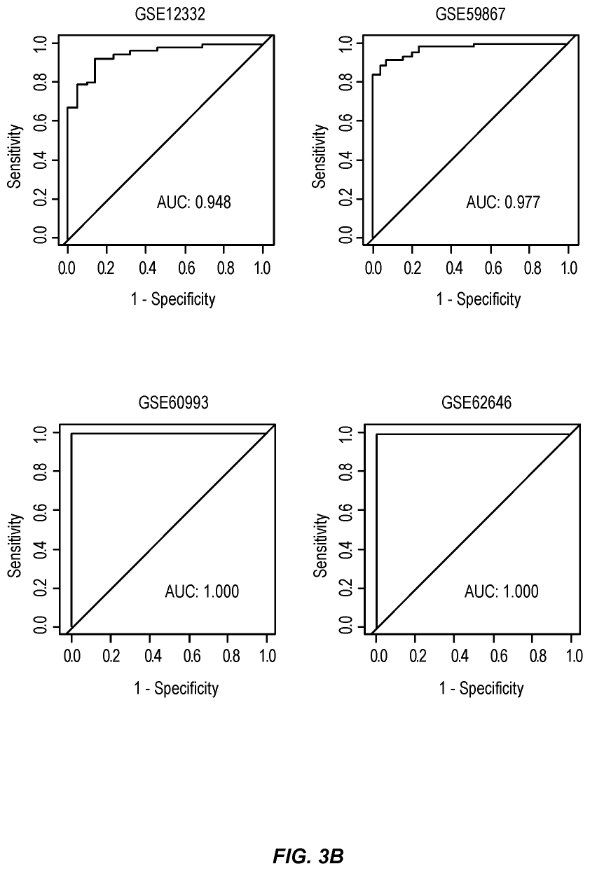

A parsimonious set of mRNA signature: The greedy forward search using the 480 mRNAs set as input yielded an optimized set of 15 mRNAs which can be more amenable for translating to clinical use in a clinically deployable platform. The forward search mRNA signature has 10 over-expressed mRNAs and 5 under-expressed mRNAs. The geometric mean value based on the 15 mRNAs effectively distinguishes MI samples from control samples (FIG. 3A). The corresponding AUROCs based on the value showed excellent discriminating power in differentiating MI samples from controls (FIG. 3B).

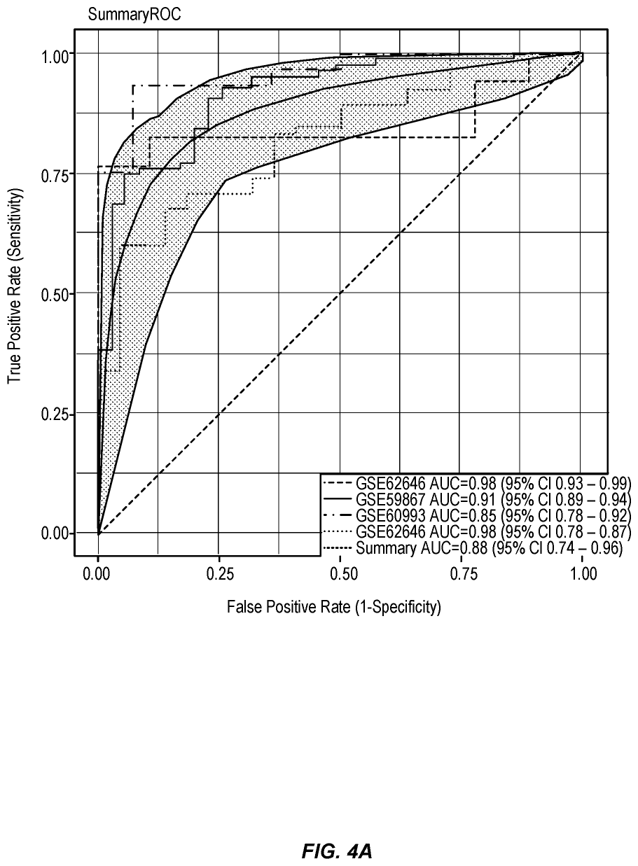

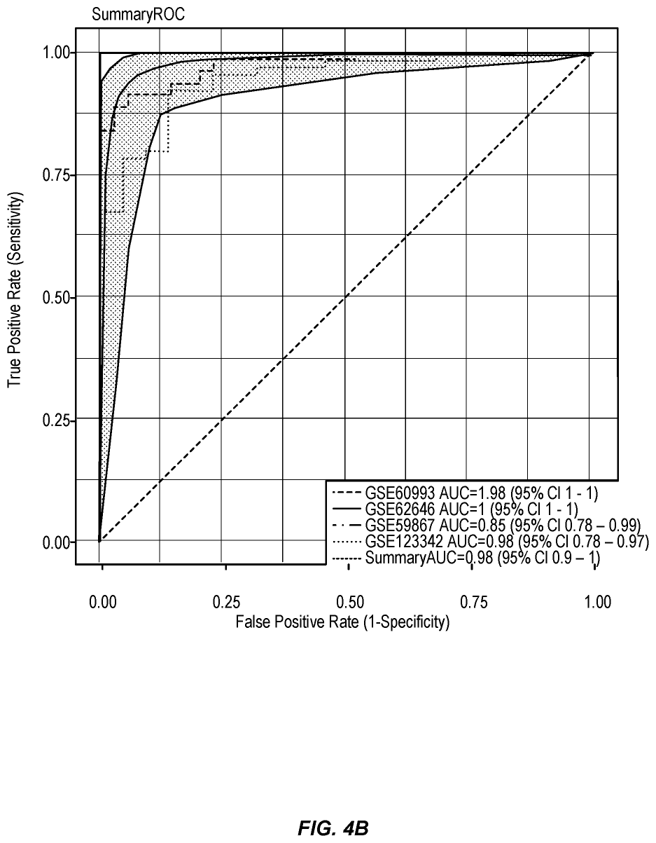

Performance summary of mRNA biomarker set: The classifier performance based on the 480 signature mRNAs and the 15 forward search mRNAs are summarized in FIGS. 4A and 4B. The resultant AUC of the 15 mRNAs was found to reach a higher level with a more robust performance (0.98 over the 4 datasets in FIG. 4B) than those with the 480 signature mRNAs (FIG. 4A). In FIGS. 4A and 4B, ROCs and AUCs were given not only for each of the 4 datasets individually, but also for the pooled results (average and 95% confident interval).

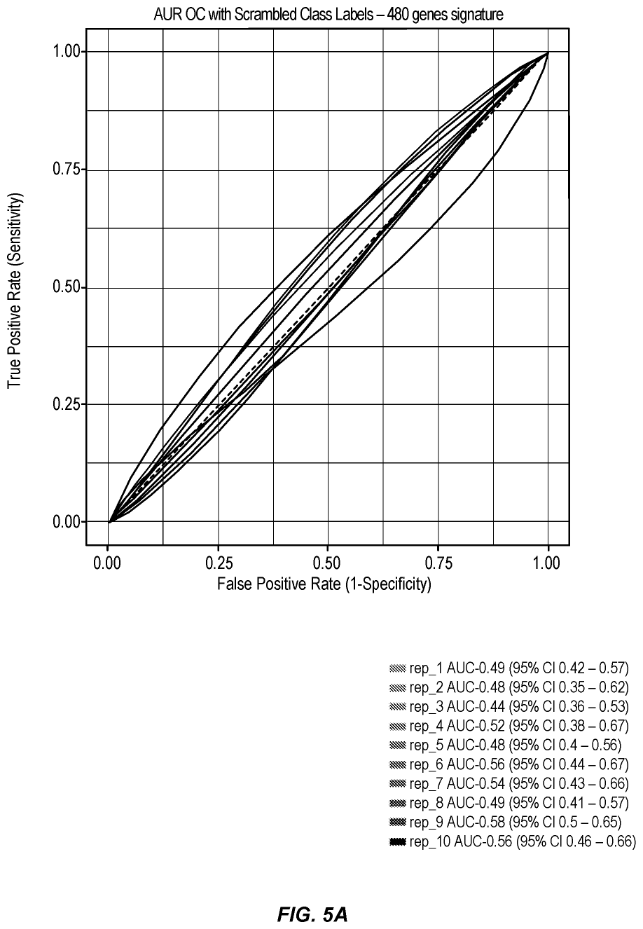

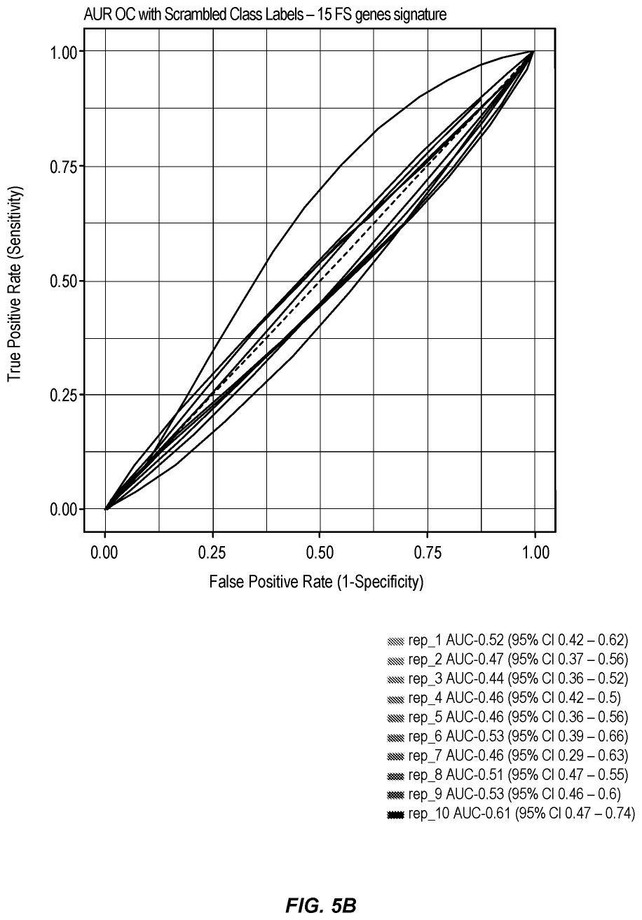

Specificity of mRNA biomarker set: Finally, ROCs and corresponding AUCs based on either the 480 mRNA signature (A) or the 15 mRNAs signature (B) with 10 repetitions of randomly permuted class labels showed that our mRNA signature is specific at distinguishing MI samples from controls (FIGS. 5A and 5B). When the class labels were randomly scrambled, the performance of the gene signature (either the long list of 480 genes or the short list of 15 genes) diminished as expected, suggesting that our markers are specific to the desired labels (MI vs. non-MI).

### 5. Discussion

Acute MI is the leading cause of death among ischemic heart diseases. An early and accurate diagnosis can provide timely treatment and reduce unwanted medical expenses. A blood mRNA-based test to effectively identify MI patients is a molecular test that could address this unmet clinical need.

Using a well-established multi-cohort analysis method, a multi-mRNA signature consisting of 480 mRNAs from four available datasets was discovered and its robust performance in discriminating MI from controls was demonstrated. Additionally, using a greedy forward search algorithm, a parsimonious set of 15 mRNAs with a similar and even better performance in distinguishing MI from controls was identified. Furthermore, it is illustrated that a reasonably good performance can be achieved with any 2-mRNA pair and their further combinations drawn from the list of 480 genes included in this report.

These findings support the prospect that an accurate diagnostic test can be developed for clinical use with subsets of the identified mRNA biomarkers from blood transcriptomic profile of patients with acute MI. Such a diagnostic test, when deployed on a rapid point-of-care platform with a turnaround time of 30 minutes or less in clinics, can enable clinicians to make accurate diagnosis of MI which is often challenging when troponin measurements and ECG are inconclusive and requires a series of sequential tests/procedures that are costly and time and resource consuming. The diagnostic test envisioned here can help clinicians to make better and timely triaging decisions, save patient lives, and reduce the healthcare cost.

### VIII. Kits and Systems

### A. Kits

In one aspect, kits are provided for the determination of the likelihood of a subject undergoing MI, wherein the kits can be used to detect the biomarkers described herein. For example, the kits can be used to detect any one or more of the biomarkers described herein, which are differentially expressed in biological (e.g., whole blood or components thereof) samples from subjects that are undergoing MI and from subjects that are not undergoing MI. The kit may include one or more agents for the detection of biomarkers, a container for holding a biological sample isolated from a human subject; and printed instructions for reacting agents with the biological sample or a portion of the biological sample to detect the presence or amount of at least one biomarker in the biological sample. The agents may be packaged in separate containers. The kit may further comprise one or more control reference samples and reagents for performing a PCR, isothermal amplification, immunoassay, NanoString, or microarray analysis, e.g., reference samples from control subjects. The kit may also comprise one or more devices or implements for carrying out any of the herein devices, e.g., 96-well plates, microfluidic cartridges, single-well multiplex assays, etc.

In certain embodiments, the kit comprises agents, e.g., primers and/or probes, for measuring the levels of one or more biomarkers (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 90, 95, 100, 200, 300, or 400 biomarkers) listed in Table 2.

In certain embodiments, the kit comprises agents, e.g., primers and/or probes, for measuring the levels of one or more pairs of biomarkers (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 pairs of biomarkers) listed in Table 3.

In certain embodiments, the kit comprises agents, e.g., primers and/or probes, for measuring the levels of one or more biomarkers (e.g., 1, 2, 3, 4, 5, 6, 7, 8, 9, 10, 11, 12, 13, 14, or 15 biomarkers) selected from the group consisting of SOCS3, OVCA2, MEF2A, PRR33, SCX, AQP9, IPPK, TNFSF8, CD163, MEF2B, HINT2, ATP1B1, ZCCHC18, CXorf65, and IMP3.

In certain embodiments, the kit comprises agents, e.g., primers and/or probes, for measuring the levels of one or more biomarkers (e.g., 1, 2, 3, 4, 5, 6, 7, or 8 biomarkers) selected from the group consisting of SOCS3, SCX, AQP9, IPPK, CD163, ATP1B1, ZCCHC18, and CXorf65.

In certain embodiments, the kit comprises agents, e.g., primers and/or probes, for measuring the levels of one or more biomarkers (e.g., 1, 2, 3, 4, 5, 6, or 7 biomarkers) selected from the group consisting of OVCA2, MEF2A, PRR33, TNFSF8, MEF2B, HINT2, and IMP3.

In certain embodiments, the kit comprises a microarray or other solid support for analysis of a plurality of biomarker polynucleotides. An exemplary microarray or other support included in the kit comprises one or more oligonucleotides that hybridize to one or more biomarkers (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 90, 95, 100, 200, 300, or 400 biomarkers) listed in Table 2. An exemplary microarray or other support included in the kit comprises one or more oligonucleotides that hybridize to one or more biomarkers (e.g., 1, 2, 3, 4, 5, 6, 7, 8, 9, 10, 11, 12, 13, 14, or 15 biomarkers) selected from the group consisting of SOCS3, OVCA2, MEF2A, PRR33, SCX, AQP9, IPPK, TNFSF8, CD163, MEF2B, HINT2, ATP1B1, ZCCHC18, CXorf65, and IMP3. An exemplary microarray or other support included in the kit comprises one or more oligonucleotides that hybridize to one or more biomarkers (e.g., 1, 2, 3, 4, 5, 6, 7, or 8 biomarkers) selected from the group consisting of SOCS3, SCX, AQP9, IPPK, CD163, ATP1B1, ZCCHC18, and CXorf65. An exemplary microarray or other support included in the kit comprises one or more oligonucleotides that hybridize to one or more biomarkers (e.g., 1, 2, 3, 4, 5, 6, or 7 biomarkers) selected from the group consisting of OVCA2, MEF2A, PRR33, TNFSF8, MEF2B, HINT2, and IMP3.

The kit can be designed for use with a specific detection system or technique, such as polymerase chain reaction (PCR) (e.g., quantitative PCR (qPCR), droplet digital PCR (ddPCR), reverse transcription PCR (RT-PCR), quantitative RT-PCR (qRT-PCR)), isothermal amplification (e.g., loop-mediated isothermal amplification (LAMP), reverse transcription LAMP (RT-LAMP), quantitative RT-LAMP (qRT-LAMP)), RPA amplification, ligase chain reaction, branched DNA amplification, nucleic acid sequence-based amplification (NASBA), strand displacement assay (SDA), transcription-mediated amplification, rolling circle amplification (RCA), helicase-dependent amplification (HDA), single primer isothermal amplification (SPIA), nicking and extension amplification reaction (NEAR), transcription mediated assay (TMA), CRISPR-Cas detection, or direct hybridization without amplification onto a functionalized surface (e.g., using a graphene biosensor). In particular embodiments, the kit can be designed for use with qRT-PCR or qRT-LAMP. The kit can contain additional materials needed for the specific detection system or technique.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of determining the composite biomarker value.

### B. Measurement Systems for Detecting and Recording Biomarker Expression

In one aspect, a measurement system is provided. Such systems allow, e.g., the detection of biomarker gene expression in a sample and the recording of the data resulting from the detection. The stored data can then be analyzed as described elsewhere herein to determine the composite biomarker value of a subject. Such systems can comprise assay systems (e.g., comprising an assay device and detector), which can transmit data to a logic system (such as a computer or other system or device for capturing, transforming, analyzing, or otherwise processing data from the detector). The logic system can have any one or more of multiple functions, including controlling elements of the overall system such as the assay system, sending data or other information to a storage device or external memory, and/or issuing commands to a treatment device.

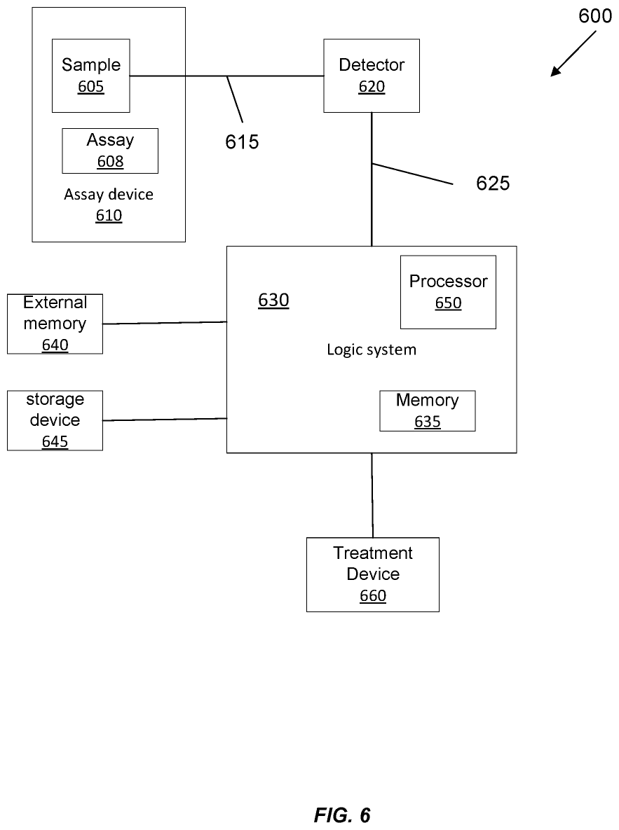

An exemplary measurement system is shown in FIG. 6. The system as shown includes a sample 605, an assay device 610, where an assay 608 can be performed on sample 605. For example, sample 605 can be contacted with reagents of assay 608 to provide a signal of a physical characteristic 615. An example of an assay device can be a flow cell that includes probes and/or primers of an assay or a tube through which a solution moves (with the solution including the assay). Physical characteristic 615 (e.g., a fluorescence intensity, a voltage, or a current), from the sample is detected by detector 620. Detector 620 can take a measurement at intervals (e.g., periodic intervals) to obtain data points that make up a data signal. In one embodiment, an analog-to-digital converter converts an analog signal from the detector into digital form at a plurality of times. Assay device 610 and detector 620 can form an assay system, e.g., an amplification and detection system that measures biomarker gene expression according to embodiments described herein. A data signal 625 is sent from detector 620 to logic system 630. As an example, data signal 625 can be used to determine expression levels for selected biomarkers. Data signal 625 can include various measurements made at a same time, e.g., different colors of fluorescent dyes or different electrical signals for different molecules of sample 605, and thus data signal 625 can correspond to multiple signals. Data signal 625, either directly or after online processing by Processor 650, may be stored in a local memory 635, an external memory 640, or a storage device 645. System 600 may also include a treatment device 660, which can provide a treatment to the subject. Treatment device 660 can determine a treatment and/or be used to perform a treatment. Examples of such treatment can include surgery, radiation therapy, chemotherapy, immunotherapy, targeted therapy, hormone therapy, and stem cell transplant. Logic system 630 may be connected to treatment device 660, e.g., to provide results of a method described herein. The treatment device may receive inputs from other devices, such as an imaging device and user inputs (e.g., to control the treatment, such as controls over a robotic system).

### C. Computer Systems and Diagnostic Systems

Certain aspects of the herein-described methods may be totally or partially performed with a computer system including one or more processors, which can be configured to perform the steps. Thus, embodiments are directed to computer systems configured to perform the steps of methods described herein, potentially with different components performing a respective step or a respective group of steps. The computer systems of the present disclosure can be part of a measuring system as described above, or can be independent of any measuring systems. In some embodiments, the present disclosure provides a computer system that calculates a composite biomarker value based on inputted biomarker expression (and optionally other) data, and determines the likelihood of a subject undergoing MI.

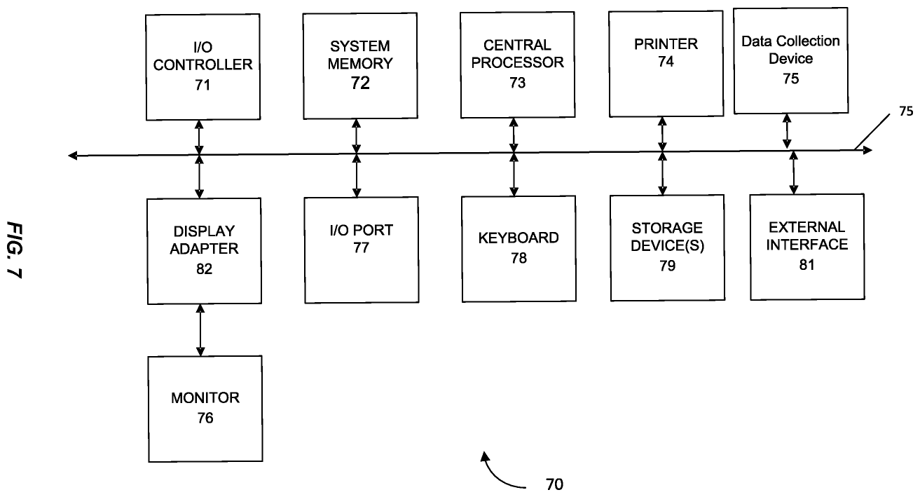

An exemplary computer system is shown in FIG. 7. Any of the computer systems may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. The subsystems shown in FIG. 7 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi, etc.) can be used to connect computer system 70 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 75, such as a camera, microphone, accelerometer, and the like. Any of the data mentioned herein can be output from one component to another component and can be output to the user. A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

In one aspect, the present disclosure provides a computer implemented method for determining the likelihood or probability of a subject undergoing MI. The computer performs steps comprising, e.g., receiving inputted patient data comprising values for the levels of one or more biomarkers in a biological sample from the patient; analyzing the levels of one or more biomarkers and optionally comparing them to respective reference values, e.g., to a housekeeping reference gene for normalization; calculating a composite biomarker value for the patient based on the levels of the biomarkers and comparing the value to one or more threshold values to assign the patient to a category (i.e., undergoing MI or not undergoing MI); and displaying information regarding the likelihood or probability of MI. In certain embodiments, the inputted patient data comprises values for the levels of a plurality of biomarkers in a biological sample from the patient, e.g., biomarkers comprising one or more biomarkers listed in Table 2 or one or more of biomarkers selected from the group consisting of SOCS3, OVCA2, MEF2A, PRR33, SCX, AQP9, IPPK, TNFSF8, CD163, MEF2B, HINT2, ATP1B1, ZCCHC18, CXorf65, and IMP3 (e.g., SOCS3, SCX, AQP9, IPPK, CD163, ATP1B1, ZCCHC18, and CXorf65; or e.g., OVCA2, MEF2A, PRR33, TNFSF8, MEF2B, HINT2, and IMP3).

In a further aspect, a diagnostic system is included for performing the computer implemented method, as described. A diagnostic system may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for determining the MI status (i.e., undergoing MI or not undergoing MI) of the subject. For example, the storage component includes instructions for calculating the composite biomarker value for the subject based on biomarker expression levels, as described herein. In addition, the storage component may further comprise instructions for performing multivariate linear discriminant analysis (LDA), receiver operating characteristic (ROC) analysis, principal component analysis (PCA), ensemble data mining methods, cell specific significance analysis of microarrays (csSAM), or multi-dimensional protein identification technology (MUDPIT) analysis. The computer processor is coupled to the storage component and configured to execute the instructions stored in the storage component in order to receive patient data and analyze patient data according to one or more algorithms. The display component displays information regarding the diagnosis of the patient. The storage component may be of any type capable of storing information accessible by the processor, such as a hard-drive, memory card, ROM, RAM, DVD, CD-ROM, USB Flash drive, write-capable, and read-only memories.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data. In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may actually comprise a collection of processors which may or may not operate in parallel. In one aspect, computer is a server communicating with one or more client computers. Each client computer may be configured similarly to the server, with a processor, storage component and instructions. Although the client computers and may comprise a full-sized personal computer, many aspects of the system and method are particularly advantageous when used in connection with mobile devices capable of wirelessly exchanging data with a server over a network such as the Internet.

The above description of example embodiments of the present disclosure has been presented for the purposes of illustration and description. It is not intended to be exhaustive or to limit the disclosure to the precise form described, and many modifications and variations are possible in light of the teaching above.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

All patents, patent applications, publications, and descriptions mentioned herein are incorporated by reference in their entirety for all purposes. None is admitted to be prior art. Where a conflict exists between the instant application and a reference provided herein, the instant application shall dominate.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “‘1’ or ‘2’ or ‘3’ or ‘1 and 2’ or ‘1 and 3’ or ‘2 and 3’ or ‘1, 2 and 3’”. Whenever a range is given in the specification, for example, a temperature range, a time range, or a composition range, all intermediate ranges and subranges, as well as all individual values included in the ranges given are intended to be included in the disclosure.

The FIGURES illustrate the architecture, functionality and operation of possible implementations of systems, methods and computer program products according to preferred embodiments, example configurations, and variations thereof. In this regard, each block in the flowchart or block diagrams may represent a module, segment, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the preferred embodiments of the invention without departing from the scope of this invention defined in the following claims.

1. A method for evaluating a patient suspected of undergoing a myocardial infarction (MI), the method comprising:
a) receiving a biological sample obtained from the patient suspected of undergoing the MI;
b) measuring one or more expression levels of at least one biomarker selected from Table 2; and
c) determining whether the patient is undergoing the MI based on the one or more expression levels of the at least one biomarker in the biological sample.

2. The method of claim 1, wherein step b) comprises measuring the one or more expression levels of at least two biomarkers selected from Table 2.

3. The method of claim 1, wherein the at least one biomarker is selected from a group consisting of SOCS3, OVCA2, MEF2A, PRR33, SCX, AQP9, IPPK, TNFSF8, CD163, MEF2B, HINT2, ATP1B1, ZCCHC18, CXorf65, and IMP3.

4. The method of claim 3, wherein the at least one biomarker is selected from a group consisting of SOCS3, SCX, AQP9, IPPK, CD163, ATP1B1, ZCCHC18, and CXorf65.

5. The method of claim 3, wherein the at least one biomarker is selected from a group consisting of OVCA2, MEF2A, PRR33, TNFSF8, MEF2B, HINT2, and IMP3.

6. The method of claim 1, wherein step c) comprises generating a composite biomarker value based on the one or more expression levels of the at least one biomarker in the biological sample, and determining, based on the composite biomarker value, whether the patient is undergoing the MI.

7. The method of claim 6, wherein the composite biomarker value not exceeding a threshold value indicates that the patient is not undergoing the MI, wherein the threshold value is determined using training samples of patients that are determined to not be undergoing the MI by a separate testing procedure.

8. The method of claim 7, further comprising:
d) determining that the patient is not undergoing the MI by comparing the composite biomarker value to the threshold value.

9. (canceled)

10. The method of claim 7, further comprising:
discharging the patient from a clinical facility based on determining the patient is not undergoing the MI.

11. The method of claim 6, wherein the composite biomarker value exceeding a threshold value indicates that the patient is undergoing the MI and is a candidate for an additional cardiovascular diagnostic testing, a therapeutic intervention, or both, wherein the threshold value is determined using training samples of patients that are determined to be undergoing the MI by a separate testing procedure.

12. The method of claim 11, further comprising:
d) determining that the patient is undergoing the MI by comparing the composite biomarker value to the threshold value and that the patient is a candidate for the additional cardiovascular diagnostic testing, the therapeutic intervention, or both.

13. The method of claim 12, further comprising:
f) subjecting the patient to the additional cardiovascular diagnostic testing, the therapeutic intervention, or both.

14-25. (canceled)

26. The method of claim 1, wherein the biological sample is whole blood, a blood component or fraction, plasma, serum, a peripheral blood mononucleated cell (PBMC), a band cell, a neutrophil, a monocyte, a T cell, or a combination thereof.

27. The method of claim 1, wherein the one or more expression levels of the at least one biomarker is detected using polymerase chain reaction (PCR), isothermal amplification, RPA amplification, ligase chain reaction, branched DNA amplification, nucleic acid sequence-based amplification (NASBA), strand displacement assay (SDA), transcription-mediated amplification, rolling circle amplification (RCA), helicase-dependent amplification (HDA), single primer isothermal amplification (SPIA), nicking and extension amplification reaction (NEAR), transcription mediated assay (TMA), CRISPR-Cas detection, and/or direct hybridization without amplification onto a functionalized surface.

28. The method of claim 27, wherein the PCR is quantitative PCR (qPCR), droplet digital PCR (ddPCR), reverse transcription PCR (RT-PCR), or quantitative RT-PCR (qRT-PCR).

29. (canceled)

30. The method of claim 27, wherein the isothermal amplification is loop-mediated isothermal amplification (LAMP), reverse transcription LAMP (RT-LAMP), or quantitative RT-LAMP (qRT-LAMP).

31-43. (canceled)

44. A method for providing an indication for a myocardial infarction (MI) in a subject, the method comprising:
(a) measuring expression levels of one or more biomarkers selected from Table 2 or one or more biomarker pairs selected from Table 3 in a biological sample obtained from the subject;
(b) evaluating the expression levels of the one or more biomarkers to yield a composite biomarker value, wherein the composite biomarker value exceeding a threshold value indicates that the subject is undergoing the MI; and
(c) administering medical care to the subject.

45. The method of claim 44, wherein the one or more biomarkers are selected from a group consisting of SOCS3, OVCA2, MEF2A, PRR33, SCX, AQP9, IPPK, TNFSF8, CD163, MEF2B, HINT2, ATP1B1, ZCCHC18, CXorf65, and IMP3.

46. The method of claim 44, wherein the composite biomarker value has been validated in multiple cohorts.

47. The method of claim 46, wherein an area under the receiver operating characteristic (ROC) curve for the identification of subjects with MI for the composite biomarker value is at least 0.75 in an independent cohort from which the composite biomarker value was derived.
